(19) United States
(12) Patent Application Publication
(10) Pub. No.: US 2025/0013155 A1
(43) Pub. Date: Jan. 9, 2025

(54) MICROFLUIDIC POROUS MEMBRANE ELECTRICAL CELL-SUBSTRATE IMPEDANCE SENSING

(71) Applicant: THE GOVERNING COUNCIL OF THE UNIVERSITY OF TORONTO, Toronto (CA)

(72) Inventors: Oleg CHEBOTAREV, Montréal (CA); Alisa UGODNIKOV, Toronto (CA); Craig SIMMONS, Toronto (CA)

(21) Appl. No.: 18/744,433

(22) Filed: Jun. 14, 2024

Related U.S. Application Data
(60) Provisional application No. 63/521,004, filed on Jun. 14, 2023.

(51) Int. Cl. G03F 7/20; G01N 33/487; G03F 7/16

(52) U.S. Cl. CPC G03F 7/2002; G01N 33/48721; G03F 7/161

(57) ABSTRACT

Methods and devices for microfluidic porous membrane electrical cell-substrate impedance sensing (ECIS) are provided. Efficient methods of fabricating ECIS membrane electrodes as well as ECIS membrane electrodes having lower electrode resistance values, which allow for higher sensitivity measurements.

### CROSS-REFERENCE TO RELATED APPLICATION(S)

This patent application claims priority from U.S. provisional patent application 63/521,004, filed Jun. 14, 2023, and herewith incorporated by reference in is entirety.

### TECHNICAL FIELD

This disclosure generally relate to cell culture platforms with electrical sensing and their fabrication.

### BACKGROUND OF THE ART

Biological barriers play a key role in maintaining homeostasis and protection against infection, with tissue-specific endothelial or epithelial cells working in tandem with support cells and matrices to control transport of substances into compartments of the body. Beyond maintaining concentration gradients and excluding pathogens, biological barrier function is also critical in the absorption and distribution of pharmaceutical agents within different tissues.

The most common in vitro model of biological barriers is the porous cell culture insert (e.g., Transwell insert), in which endothelial or epithelial barrier cells are grown on a permeable membrane that can be placed in a traditional cell culture well plate to create a cell monolayer as a barrier with luminal and abluminal compartments. This is a simple, robust method for emulating the multicellular environment of biological barriers. The blood-brain barrier (BBB), for example, is frequently modeled in this setup, enabling investigation of the effects of conditioned medium and various arrangements of supporting cells (e.g., astrocytes, pericytes) in direct or indirect contact with endothelial cells grown on porous cell culture inserts.

Evaluation of barrier function in porous cell culture inserts often involves measurement of the diffusion of a molecular tracer (e.g., FITC-dextran) to determine barrier permeability. However, the dyes used in these assays may themselves interfere with molecular transport function and barrier integrity, thus motivating the use of electrical sensing as a non-invasive alternative. The most common electrical sensing technique for biological barriers is transepithelial/endothelial electrical resistance (TEER), which measures conductance of ions across paracellular junctions through the application of an alternating current by electrodes placed on either side of the membrane. In the standard TEER setup, AC current at a frequency of 12.5 Hz—selected to avoid charging the electrode and/or cell layer—is applied across the cell layer to determine barrier resistance. Because TEER calculations include normalization to culture area, it is theoretically possible to compare values between various barrier types and across different culture setups.

While TEER remains a gold standard for assessing the integrity of barrier models, the typical handheld “chopstick” electrode TEER device requires removal of the cultures from the incubator to test each well individually, which can disrupt the cell layers. Additionally, variations in electrode positioning hinder reproducibility of TEER measurement due to the non-uniform electric field created by the chopstick electrodes. Systems that integrate the electrodes directly into custom culture platforms or into microfluidic devices for microphysiological barrier modelling address the challenges of manual handling and electric field uniformity to facilitate non-invasive, real-time monitoring. However, because TEER electrodes are placed on surfaces above and below the culture substrate, they measure bulk impedance of everything between the electrodes, not just the cell monolayer. As such, TEER measurement of cell monolayer barrier function is confounded in culture systems that contain multiple cell types or biomaterials (e.g., hydrogels), including most BBB models and microphysiological models.

An alternative to TEER is electrical cell-substrate impedance sensing (ECIS). ECIS applies similar principles as TEER except the electrodes are integrated into the substrate on which the cells are grown. This integration into the substrate allows cells to attach and proliferate directly on the electrodes, enabling more localised and sensitive impedance measurements specifically of the cell barrier compared to TEER. Furthermore, depending on the electrode placement and size, ECIS devices enable impedance measurements at higher electrical frequencies (e.g., 40 kHz), which can be used to determine additional characteristics of the cell layer (i.e., cell proliferation, cell viability, cell function, etc.). The major disadvantage with current ECIS systems, however, is the limited compatibility of cell culture substrates with ECIS electrodes; current ECIS devices resemble standard printed circuit boards and require electrodes to be printed on either glass or stiff plastic.

Cell culture models of endothelial and epithelial barriers typically use porous membrane inserts (e.g., Transwell inserts) as a permeable substrate on which barrier cells are grown, often in co-culture with other cell types on the opposite side of the membrane.

In order to implement ECIS capabilities in porous membrane systems typical for barrier modeling, alternative electrode printing strategies are required; this has proven to be challenging, forcing the use of expensive and custom-made devices (i.e., using e-beam evaporation to deposit gold onto the membrane (Gangopadhyay et al., 2017; Ramiah Rajasekaran et al., 2020)) that are impractical for large scale commercial manufacture. Moreover, these alternative printing strategies can result in high electrode impedance values that can drown out ECIS measurements.

### SUMMARY

In accordance with a first aspect, there is provided a method of manufacturing an electrical cell-substrate impedance sensing (ECIS) membrane electrode comprising: heat-bonding a biocompatible electrically conductive sheet, preferably a gold sheet or gold alloy sheet, directly to a porous membrane to form a laminate having an electrically conductive surface and a porous membrane surface; and forming electrode components by etching the electrically conductive surface using photolithography techniques.

In one embodiment, this method includes forming the electrode components using photolithography techniques comprising:
a) hot embossing the gold- or gold alloy-coated membrane to a rigid or substantially rigid substrate;
b) applying photoresist film over the gold- or gold alloy-coated membrane, preferably a dry film photoresist, more preferably applying a dry film photoresist by lamination at 20-25° C.;
c) placing a photomask on top of the photoresist, and exposing the photoresist to UV light, preferably exposing the photoresist to UV light for 8-10 seconds;
d) removing unexposed photoresist, optionally using 1% sodium carbonate in water solution, and washing, optionally with isopropanol, to remove any residue;
e) using gold etching solution, preferably TFA gold etch, to remove gold/gold alloy not covered by the exposed photoresist, and washing, optionally with acetone, to remove etching solution and photoresist; and
optionally, f) removing the remainder of the photoresist, optionally using 0.5 M NaOH.

In a further aspect, there is provided a method of manufacturing an ECIS membrane electrode comprising:
(a′) Laminating a thermoplastic film, preferably a wax and polyolefin blend, more preferably Parafilm™ to a substrate, preferably a thermoplastic substrate, more preferably a flexible thermoplastic substrate;
(b′) Laminating porous membrane coated in gold to the thermoplastic film layer of the laminate of step (a′), optionally wherein (a′) and (b′) are performed concurrently;
(c′) Hot embossing the laminate of step (b′) at a temperature and pressure such that the porous membrane and thermoplastic film reversibly adhere;
(d′) Forming electrode components by etching the electrically conductive surface using photolithography techniques; and
(e′) Removing the porous membrane and electrode components from the thermoplastic-substrate layer.

Also provided are ECIS membrane electrodes produced by the methods provided herein.

In one aspect, there is provided an electrical cell-substrate impedance sensing (ECIS) membrane electrode comprising a biocompatible porous membrane having deposited thereon a biocompatible electrode comprising a working electrode and a counter electrode, wherein the working electrode and counter electrode are configured to be positioned in a microfluidic channel having a width of 50 μm to 5000 μm, preferably 125 μm-3000 μm and a height of 50 μm-3000 μm, preferably 100 μm-500 μm.

In the methods and/or the ECIS membrane electrodes provided:
the porous membrane may be formed from a polyester, polycarbonate, polytetrafluoroethylene (PTFE) and/or silicon, preferably polyethylene terephthalate (PETE); and/or
the porous membrane may have a pore size between 0.1 μm and 20 μm, preferably between 0.1 μm and 10 μm; and/or
the electrodes may be formed of gold or a biocompatible gold alloy, optionally an alloy with silver and/or copper; and/or
the electrodes each may have a diameter between 125 μm and 2.5 mm, preferably between about 250 μm and 2 mm; and/or
multiple working electrodes may be operatively connected in parallel to a common counter electrode; and/or
the working electrodes may each have a diameter≤500 μm, preferably ≤250 μm, more preferably between 125 μm and 250 μm.

Also provided in an apparatus for measuring cell-electrode impedance in biological cells, comprising: an ECIS membrane electrode as provided herein; and an upper supporting plate having defined thereon an upper cell-culture channel, the upper supporting plate being secured to a first side of the ECIS membrane electrode and operable to hold a cell-culture medium; and a lower supporting plate having defined thereon a lower cell-culture channel operable to hold a cell-culture medium, the lower supporting plate being secured to a second side of the ECIS membrane electrode.

In some embodiments, the upper cell-culture channel is operatively connected to a fluid inlet or a fluid outlet, preferably a fluid inlet and a fluid outlet, in the upper or lower supporting plate preferably the upper supporting plate; and/or the lower cell-culture channel is operatively connected to a fluid inlet or a fluid outlet, preferably a fluid inlet and a fluid outlet in the lower supporting plate, in the upper or lower supporting plate preferably the upper supporting plate. The ECIS membrane electrode may be secured to one or both of the upper supporting plate and the lower supporting plate by two-sided tape. The walls of one or both of the upper cell-culture channel and the lower cell-culture channel may be defined by an aperture in the two-sided tape, preferably, coincident apertures in multiple layers of two-sided tape affixed between the ECIS membrane and the upper supporting plate or lower supporting plate.

In some embodiments, the upper cell-culture channel and the lower cell-culture channel have a width between 50 μm and 5000 μm, preferably 125 μm and 3000 μm and a height of between 50 μm and 3000 μm, preferably 100 μm-500 μm.

The apparatus suitably further includes circuitry electrically coupling the working and counter electrode to a device for producing alternating current and a device for measuring impedance signal output, optionally a lock-in amplifier.

In some embodiments, the working electrode, preferably an array of working electrodes, are positioned in a central region of the channel spaced from the inlet and outlet.

Also provided is a system comprising an apparatus as provided herein and a digital signal processor that collects and stores values, optionally comprising one or more of input/output voltages, impedance, resistance, capacitance and phase angle.

Also provided is a method comprising providing an apparatus or system as described herein and measuring electrical impedance between the working and counter electrode.

Also provided is a method of imaging cells comprising culturing the cells in the upper cell-culture channel or the lower cell-culture channel of an apparatus or system as provided herein and imaging the cells through one or both of the upper supporting plate and the lower supporting plate.

Also provided is a method of co-culturing cells comprising providing an apparatus or system as described herein and culturing a first cell type in the upper cell-culture channel and culturing a second cell type in the lower cell-culture channel.

Also provided is a method of forming a biological barrier model for analysis comprising culturing one or more cell types in an apparatus or system as provided herein under conditions enabling biological barrier assembly, optionally wherein the one or more cell types are endothelial and/or epithelial cells.

Also provided is a method of assessing cell coverage and/or barrier integrity comprising culturing one or more cell types in an apparatus or system as provided herein and measuring impedance between one or more working electrodes and one or more counter electrodes over time and deriving change in impedance value(s) or value(s) derived from the impedance value(s) over time, wherein increased impedance indicates increased cell coverage and/or increased barrier integrity; and/or measuring impedance between one or more working electrodes and one or more counter electrodes over time and comparing impedance value(s) or value(s) derived from impedance values in a comparative model having known cell coverage and/or barrier integrity.

Many further features and combinations thereof concerning the present improvements will appear to those skilled in the art following a reading of the instant disclosure.

### DESCRIPTION OF THE DRAWINGS

FIGS. 1A to 1D show elements of a microfluidic ECIS platform according to embodiments.

FIG. 1A shows a cross-sectional schematic of a microfluidic ECIS membrane electrode platform, according to an embodiment.

FIG. 1B shows a top-down view of a fabricated device, according to an embodiment.

FIG. 1C shows device components, according to an embodiment.

FIG. 1D shows a channel view of working electrodes (d=250, 500, 750 μm) and counter electrode (CE), according to an embodiment.

FIGS. 2A to 2E show an example of porous membrane electrode fabrication, and assembly into porous membrane ECIS (PM-ECIS) devices.

FIG. 2A shows gold leaf sheets were bonded to polyethylene terephthalate porous membranes using a pressured thermal bonding process, followed by photolithographic patterning and etching to form electrodes.

FIG. 2B shows fabrication of a custom 6-well “Transwell-like” insert incorporating PM-ECIS.

FIG. 2C shows application of electrical insulating masks on both sides of the porous membrane electrodes.

FIG. 2D shows fabrication of a custom 96-well device incorporating PM-ECIS.

FIG. 2E is a photograph of a 6-well plate with three 6-well PM-ECIS inserts with flat flex cable gold finger connectors.

FIGS. 2F to 2J compare gold leaf hot embossed as employed in the process shown in FIG. 2A vs. e-beam deposited gold onto porous membrane.

FIG. 2F is a scanning electron microscope image of gold leaf hot embossed onto porous membrane (X500 SE). Hot embossed gold leaf forms a continuous metal layer that spans the pores in the membrane, which is left intact after UV lithography using TFA gold etch solution.

FIG. 2G is a scanning electron microscope image of gold leaf hot embossed onto porous membrane (X2000 SE). Hot embossed gold leaf forms a continuous metal layer that spans the pores in the membrane, which is left intact after UV lithography using TFA gold etch solution.

FIG. 2H is a scanning electron microscope image of e-beam deposited gold onto porous membrane (X500 SE). Applying the same etching process to the e-beam deposited gold membrane yields a non-uniform metal layer with heterogeneous coverage of the membrane and discontinuities at the pores.

FIG. 2I is a scanning electron microscope image of e-beam deposited gold onto porous membrane (X1000 SE). Applying the same etching process to the e-beam deposited gold membrane yields a non-uniform metal layer with heterogeneous coverage of the membrane and discontinuities at the pores.

FIG. 2J is a scanning electron microscope image of e-beam deposited gold onto porous membrane (X 5000 SE). Applying the same etching process to the e-beam deposited gold membrane yields a non-uniform metal layer with heterogeneous coverage of the membrane and discontinuities at the pores.

FIG. 2K shows resistivities measured for sputter-coated and gold leaf hot embossed electrodes, demonstrating significantly lower resistivity of the gold leaf hot embossed electrodes (p=0.015).

FIG. 3 shows high fidelity patterning of different electrode designs on PM-ECIS devices. Schematics (left) and photographs (right) of (A, B) single electrode design for 6-well insert; (C, D) six-finger electrode design for 6-well insert; and (E, F) interdigitating eight-finger electrode design for 96-well plate. (G) Scanning electron micrograph of gold electrode patterned as the single electrode design on a porous membrane, showing high fidelity patterning and adhesion. (H) Scanning electron micrograph of interface of a patterned electrode and bare porous membrane, showing no deformation or clogging of the nominal 3 μm pores on the bare membrane.

FIG. 4 shows PM-ECIS devices support endothelial cell culture. Human umbilical vein endothelial cells (HUVECs) stained with calcein AM and ethidium homodimer to indicate live and dead cells, respectively, showed high viability when grown on (A) gold electrodes (indicated by white dotted line) and the adjacent porous membrane and (B) areas of the membrane with the tape mask, comparable to HUVECs grown on (C) standard tissue culture-treated polystyrene plates. Scanning electron micrographs of 4 day cultures confirmed HUVEC adhesion to and coverage of (D) finger-like and (E) round electrodes, with (F) the cell layer spanning the interface of the electrode and bare membrane, with no visible difference in cell morphology. Scale bars in A-C=100 μm.

FIGS. 5A to 5D show PM-ECIS measurement of endothelial monolayer resistance is sensitive to seeding density.

FIG. 5A shows representative traces of 4 kHz PM-ECIS measurement of HUVECs initially seeded at densites of 250,000 cells/cm² or 100,000 cells/cm² in 96-well PM-ECIS devices. Dotted lines show traces from a separate device under the same conditions. Also shown are cell-free control devices.

FIG. 5B shows PM-ECIS resistances normalized to cell-free controls across the three measured frequencies (400, 4000, 40000 Hz) at end of day 4 for the high and low density cultures.

FIG. 5C shows representative traces of 4 kHz PM-ECIS resistances measured for HUVECs cultured in 6-well devices with either a single electrode or 6-finger electrode. Dotted lines show traces from a separate device under the same conditions. Also shown are cell-free control devices.

FIG. 5D shows PM-ECIS resistances normalized to cell-free controls across the three measured frequencies (400, 4000, 40000 Hz) at end of day 4 for the 6-well single electrode devices with and without HUVECs. N=3 devices.

FIG. 6 shows PM-ECIS devices support co-culture of endothelial cells and astrocytes. (A) Immortalised primary human brain microvascular endothelial cells (HBMVECs) grown in monoculture on the apical side of 6-well PM-ECIS devices after 4 days in culture, stained for ZO-1 (white triangles indicate example expression of ZO-1 at the peripheries of cells) and nuclei (Hoescht dye). Scale bar=100 μm. (B) Human astrocytes grown in monoculture on the underside of the PM-ECIS device, stained for glial fibrillary acidic protein (GFAP) and nuclei. (C) Confocal maximum intensity projection of apical side HBMVECs co-cultured with basolateral side astrocytes, demonstrating phenotypic ZO-1 and GFAP staining. Nuclei stained by Hoescht. (D) PM-ECIS resistance measurement at 4000 Hz for 2.5 days of culture, showing endothelial layer resistance was unaffected by the presence of co-culture astrocytes on the opposite of the membrane.

FIG. 7 shows pericellular resistances and cell membrane capacitance can be derived from PM-ECIS measurements. HUVECs grown in 6-well devices were treated with serum-containing medium hour 10 (arrows) and monitored continuously by PM-ECIS. The network model developed by Giaever and Keese (1991) was applied to derive (A) cell-cell pericellular resistance, Rb; (B) cell-electrode pericellular resistance, a; and (C) cell layer membrane capacitance, Cm. (D) 4 kHz PM-ECIS resistance measurements of HUVECs treated at t=0 (arrow) with media only (HUVECs) or media+thrombin (HUVECs+thrombin) to cause barrier disruption. (E) Calculated pericellular resistance, Rb, for thrombin-disrupted HUVEC layer.

FIGS. 8A to 8F show endothelial monolayer resistances measured by PM-ECIS correlate with permeability measurements. Primary human brain microvascular endothelial cells (HBMECs) were seeded on 6-well PM-ECIS devices at initial (day 0) seeding densities of 5,000 cells/cm² or 30,000 cells/cm², and 10 kDa FITC-dextran permeability and PM-ECIS measurements performed on day 1, 2, 3, and 4.5 post-seeding (indicated by arrows). PM-ECIS was measured continuously throughout the experiment, with measurements made at 400 Hz, 4 kHz, and 40 kHz. N=3 devices initially seeded at 5,000 cells/cm² and N=4 devices initially seeded at 30,000 cells/cm².

FIG. 8A shows PM-ECIS measurements made at 400 Hz.

FIG. 8B shows PM-ECIS measurements made at 4 kHz.

FIG. 8C shows PM-ECIS measurements made at 40 kHz.

FIG. 8D shows final (day 4.5) PM-ECIS resistances measured at 400 Hz were significantly correlated with FITC-dextran permeabilities measured in the same devices.

FIG. 8E shows final (day 4.5) PM-ECIS resistances measured at 4 kHz were significantly correlated with FITC-dextran permeabilities measured in the same devices.

FIG. 8F shows final (day 4.5) PM-ECIS resistances measured at 40 kHz were significantly correlated with FITC-dextran permeabilities measured in the same devices.

FIGS. 9A to 9D show fabrication and setup of 6-well ECIS membrane electrode devices per Example 3.

FIG. 9A shows the fabrication process involving thermal bonding of gold leaf to membrane and UV lithography patterning of electrodes. The membrane electrodes are then assembled into a cell-culture insert format. Arrow in bottom schematic shows flow of current between ECIS electrodes through the endothelial cell layer and culture medium.

FIG. 9B shows electrode design for testing three working electrode sizes of 250, 500, and 750 μm diameter. In the inset, hatched grey denotes exposed electrode areas as defined by a tape mask; solid grey denotes electrode traces covered by the tape mask. Counter electrode=2 mm diameter. Edge-to-edge working electrode-counter electrode distance=1.5 mm.

FIG. 9C shows a top-down view of a membrane electrode device.

FIG. 9D shows PM-ECIS devices connected to a 24-channel multiplexer, which in turn are connected to a lock-in amplifier and computer.

FIG. 10 shows ECIS membrane device supports growth of confluent endothelial monolayer. (A) Fluorescence microscopy images of human umbilical vein endothelial cells (HUVECs) covering counter electrode, stained for VE-cadherin (yellow) and nuclei visualized with Hoechst 33342 (blue). Scale bar=200 μm. (B) Confocal microscopy imaging shows expression of VE-cadherin (yellow) and ZO-1 (magenta) in HUVECs grown on electrode surface. Nuclei visualized with Hoechst 33342. Scale bars=50 μm. (C) Confocal microscopy imaging shows HUVEC monolayer spanning the electrode-membrane boundary. Scale bar=20 μm. (D) Representative frequency vs. resistance plot for Day 4 post-confluence HUVECs grown on 250 μm electrodes; CTRL=cell-free control. Each line corresponds to a replicate in a separate cell culture insert. (E) HUVEC normalized resistance (Rcell/Rcell-free) for three electrode sizes (d=250, 500, 750 μm) across frequencies. N=5-7 cell-covered, 6-7 cell-free for each electrode size. (F) Caco-2 normalized resistance values across the scanned frequencies. Normalized resistance (Rcell/Rcell-free) for three electrode sizes (d=250, 500, 750 μm) across frequencies.

FIGS. 11A to 11D show electrode size-dependent response in impedance and resistance for HUVEC monolayer grown in PM-ECIS devices. N=5-7 cell-covered and 6-7 cell-free electrodes for each electrode size. *p<0.001 using one-way ANOVA with Bonferroni test.

FIG. 11A shows representative ΔR (4 kHz) vs. time plot for Day 4 post-confluence HUVECs. ΔR=Rcell−Rcell-free for corresponding electrode sizes.

FIG. 11B shows normalized R values for Day 4-post confluence HUVECs grown on three electrode sizes (d=250, 500, 750 μm); ΔR normalized to 250 μm electrodes for measurements taken at 4 kHz.

FIG. 11C shows representative impedance (40 kHz) vs. time plot for Day 4 post-confluence HUVECs.

FIG. 11D shows ΔZ normalized to 250 μm electrodes for measurements taken at 40 kHz. ΔZ=Zcell−Zcell-free for corresponding electrode sizes.

FIGS. 11E to 11H show electrode size-dependent response in impedance and resistance for Caco-2 epithelial cells grown in PM-ECIS.

FIG. 11E shows resistance measurements for Caco-2 epithelial cell monolayer at 400 Hz for 750 μm electrodes showed characteristic increase over the first ˜2 days of culture, with change in resistance being inversely proportional to the electrode size.

FIG. 11F shows resistance measurements for Caco-2 epithelial cell monolayer at 400 Hz for 500 μm electrodes showed characteristic increase over the first ˜2 days of culture, with change in resistance being inversely proportional to the electrode size.

FIG. 11G shows resistance measurements at 1000 Hz for 250 μm electrodes showed similar increase over the first ˜2 days of culture

FIG. 11H shows validation by TEER measurements of the same cultures of the temporal trend in barrier resistance from FIGS. 11E-11G (on all electrode sizes, resistance values decreased from Day 3 to 5 of culture, indicating reduced barrier integrity with time in culture).

FIGS. 12A to 12L show electrode size-dependent response in impedance and resistance to barrier disruption with ethylene glycol-bis(β-aminoethyl ether)-N,N,N′,N′-tetraacetic acid (EGTA) or thrombin. Statistical analysis performed using paired t-test (pre-treatment vs. other conditions; *=p<0.05, **=p<0.01).

FIG. 12A shows representative 750 μm cell-covered and cell-free control electrodes showing change in PM-ECIS resistance at 4 kHz after treatment with 5 mM EGTA in confluent HUVEC monolayers. Black arrow indicates time EGTA was added.

FIG. 12B shows representative 500 μm cell-covered and cell-free control electrodes showing change in PM-ECIS resistance at 4 kHz after treatment with 5 mM EGTA in confluent HUVEC monolayers. Black arrow indicates time EGTA was added.

FIG. 12C shows representative 250 μm cell-covered and cell-free control electrodes showing change in PM-ECIS resistance at 4 kHz after treatment with 5 mM EGTA in confluent HUVEC monolayers. Black arrows indicate time EGTA was added.

FIG. 12D shows resistance pre-EGTA treatment, 15 min post-treatment, and 120 min post-treatment for 750 μm electrodes. N=3-4 cell-covered electrodes.

FIG. 12E shows resistance pre-EGTA treatment, 15 min post-treatment, and 120 min post-treatment for 500 μm electrodes. N=3-4 cell-covered electrodes.

FIG. 12F shows resistance pre-EGTA treatment, 15 min post-treatment, and 120 min post-treatment for 250 μm electrodes. N=3-4 cell-covered electrodes.

FIG. 12G shows representative 750 μm cell-covered and cell-free control electrodes showing change in resistance at 4 kHz after treatment with 1 U/mL thrombin. Black arrow indicates time thrombin was added.

FIG. 12H shows representative 500 μm cell-covered and cell-free control electrodes showing change in resistance at 4 kHz after treatment with 1 U/mL thrombin. Black arrow indicates time thrombin was added.

FIG. 12I shows representative 250 μm cell-covered and cell-free control electrodes showing change in resistance at 4 kHz after treatment with 1 U/mL thrombin. Black arrow indicates time thrombin was added.

FIG. 12J shows resistance pre-thrombin treatment, at minimum value post-treatment, and at t=120 min post-treatment for 750 μm electrodes. N=3 cell-covered electrodes.

FIG. 12K shows resistance pre-thrombin treatment, at minimum value post-treatment, and at t=120 min post-treatment for 500 μm electrodes. N=3 cell-covered electrodes.

FIG. 12L shows resistance pre-thrombin treatment, at minimum value post-treatment, and at t=120 min post-treatment for 250 μm electrodes. N=3 cell-covered electrodes.

FIG. 13 shows comparison of ECIS membrane method vs. TEER. Correlation between 4 kHz ECIS resistance and TEER resistance across varying concentrations of NaCl solution (0.005, 0.01, 0.1, 1M) for 250, 500, and 750 μm working electrodes. Each data point represents mean for N=7-8 electrodes. Pearson correlation analysis with trendlines from linear regression for illustrative purposes.

FIG. 14 shows comparison of ECIS membrane method vs. TEER. (A, B, C) Correlation between normalized TEER and ΔR for HUVEC across range of timepoints (6, 24, 48 h). ΔR=Rcell−Rcell-free; N=2-3 cell-covered and 6-8 cell-free electrodes per timepoint, for each electrode size. ΔR and TEER normalized to 6 h time point. (A) 250 μm working electrodes. (B) 500 μm working electrodes. (C) 750 μm working electrodes. Pearson correlation analysis with trendlines from linear regression for illustrative purposes.

FIGS. 15A to 15D show the effect of collagen hydrogel on ECIS resistance vs. chopstick TEER.

FIG. 15A shows the measurement setup for ECIS resistance and chopstick TEER per Example 4.

FIG. 15B shows resistance values at 4 kHz for devices with collagen gel in bottom channel vs. control for ECIS at Day 1, N=2-4 per condition. Unpaired t-test, * p<0.01. C.

FIG. 15C shows resistance values at 4 kHz for devices with collagen gel in bottom channel vs. control for TEER at Day 1, N=2-4 per condition. Unpaired t-test, * p<0.01. C.

FIG. 15D shows change in resistance values at 4 kHz for devices with collagen gel in bottom channel vs. control 2 hours post-polymerization (t=2.5 h) for various working electrode diameters (ΔR=R2h post-polymerization−Rt=0); N=3-5 per condition.

FIGS. 16A to 16F show results from a blood-brain barrier model in microfluidic ECIS platform.

FIG. 16A is a schematic of a blood-brain barrier (BBB) co-culture setup, with HBMEC in top channel and primary human astrocytes (AC) in bottom channel.

FIG. 16B and FIG. 16C show fluorescence microscopy imaging from top side, showing HBMEC cell layer on counter electrode surface (black), and AC embedded in hydrogel underneath.

FIG. 16D and FIG. 16E show confocal imaging of BBB co-culture in microfluidic setup from astrocyte side of membrane. Dotted line indicates border between working electrode and membrane. AC stained for GFAP (green), HBMEC stained for ZO-1 (pink) and nuclei visualized with Hoechst 33342.

FIG. 16F shows ECIS measurements of BBB co-culture over 5 days in static condition, showing traces for three different co-culture devices. Cell seeding occurred at t=0; black arrows=media changes. Mean±SD plotted for N=3 independent electrodes (d=500 μm) for co-culture condition.

FIGS. 17A and 17B show microfluidic PM-ECIS measurements upon application of fluid flow per Example 5.

FIG. 17A shows resistance values measured at 4 kHz in blank devices filled with PBS before (static) and after application of fluid flow at 1.3 mL/min (equivalent to a wall shear stress of 5 dyn/cm²).

FIG. 17B shows no significant differences between the pre-flow and post-flow values for any of the electrode sizes (ΔR=Rpost-flow−Rpre-flow). N=4 for each electrode size.

FIGS. 18A to 18C show ECIS measurements of HUVECs in perfusion culture.

FIG. 18A shows live fluorescence imaging of GFP-expressing HUVECs perfused for up to 5 days confirming viability and adherence of cells to porous membrane and electrodes vs. cells grown under static conditions for the same duration.

FIG. 18B shows immunostaining for ZO-1 and VE-cadherin and imaging with confocal microscopy confirmed presence of confluent monolayer on working electrodes.

FIG. 18C shows immunostaining for ZO-1 and VE-cadherin and imaging with confocal microscopy confirmed presence of confluent monolayer on counter electrodes.

FIGS. 19A to 19F show ECIS measurements of HUVECs in perfusion culture. Cell seeding at t=0; black inverted triangle indicates manual media change on Day 1; t=syringes refilled with culture medium. *=p<0.05, **=p<0.01, ***=p<0.001.

FIG. 19A, left panel shows impedance measured at 40 kHz over 5 days of culture, 3 days of perfusion. Mean±SD plotted for N=3 electrodes per condition, 750 μm working electrodes (d=750 μm). Stars indicate timepoints for impedance comparisons. FIG. 19A right panels shows a comparison of impedance measured immediately before starting perfusion culture, minimum impedance value reached following start of perfusion, impedance after 1.5 days of flow, and 3 days of flow (ΔZ=Ztimepoint−Zpre-cell seeding baseline).

FIG. 19B shows impedance at 40 kHz, d=500 μm; N=2-3 electrodes per condition. Mean±SD plotted where applicable; for time intervals with N=2, individual replicates shown.

FIG. 19C show impedance at 40 kHz, d=250 μm; N=2-3 electrodes per condition. Mean±SD plotted where applicable; for time intervals with N=2, individual replicates shown.

FIG. 19D shows resistance measured at 4 kHz over 5 days of culture, 3 days of perfusion. Mean±SD plotted for N=3 electrodes per condition, d=750 μm.

FIG. 19E shows resistance measured at 4 kHz over 5 days of culture, 3 days of perfusion. Mean±SD plotted for N=3 electrodes per condition, d=500 μm.

FIG. 19F shows resistance measured at 4 kHz over 5 days of culture, 3 days of perfusion. Mean±SD plotted for N=3 electrodes per condition, d=250 μm.

FIGS. 20A to 20D show the 250 μm electrodes were the most sensitive to changes in barrier resistance, both temporally and with respect to magnitude change in resistance.

FIG. 20A shows all three of the electrode sizes detected a significant decrease in resistance compared to static control at the post-flow minimum, with 250 μm electrodes showing the greatest magnitude decrease compared to 500 μm (p<0.01) and 750 μm (p<0.001).

FIG. 20B shows the 250 μm electrodes were most sensitive to immediate change in barrier integrity following flow, with a significant decrease in resistance at 4 kHz (p=0.039) compared to static control detected within 30 minutes of applying fluid flow.

FIG. 20C shows that for 500 μm electrodes decreased resistance was not detected until 1.5 hours post-flow (p=0.034).

FIG. 20D shows that for 750 μm electrodes decreased resistance was not detected until 1.5 hours post-flow (p=0.026).

### DETAILED DESCRIPTION

The present disclosure provides a cell culture platform with integrated real-time electrical cell-substrate impedance sensing (ECIS), and methods for fabrication of electrodes for the device, as well as the device itself.

Also provided herein is a cost-effective method to adapt ECIS technology to porous substrate-based in vitro models. The disclosed processes enable high fidelity patterning of electrodes on porous membranes that can be incorporated into well plates of various sizes. The porous membrane ECIS (PM-ECIS) disclosed herein provides excellent cell biocompatibility with mono- and co-culture set ups, and can enable sensitive, real-time measurement of changes in endothelial cell barrier impedance with cell growth and barrier disruption. As detailed in the Examples, the system was validated by direct correlation of PM-ECIS impedance and permeability coefficients obtained from molecular tracer permeability assays. Integration of ECIS into conventional porous cell culture inserts provides a versatile, sensitive, and automated alternative to TEER to measure barrier function in vitro.

The cell culture platform comprises biocompatible, preferably gold leaf of gold-alloy electrodes, integrated onto a porous membrane, allowing for monitoring of cells grown in co-culture set up. Embodiments include Transwell-like cell culture inserts for static culture in microwell plates.

Fluid shear is known as a critical factor in recapitulating physiological microenvironments in models such as blood vessels, blood-tissue barriers, and intestinal epithelium. Provided herein is a novel PM-ECIS platform that combines microfluidic architecture to create a microenvironment suitable for modeling tissues where fluid shear plays a role in physiological function, as well as integrated ECIS electrodes on porous membranes to allow for co-culture and simultaneous non-invasive, direct monitoring of cell monolayers.

As used herein, “ECIS membrane electrodes” means a non-conducting polymer sheet with pores traversing the thickness of the sheet (also referred to herein as a porous membrane) on which electrodes or electrode structure units are fabricated or incorporated.

### Porous Membrane

The porous membrane should be biocompatible, which in this context can be understood to mean formed of a material compatible with culturing cells or tissues of interest so as to enable analyses of interest.

Non-limiting examples of membrane materials are polyester, polycarbonate, polytetrafluoroethylene (PTFE) and silicon. In some embodiments, the membrane is formed of polyethylene terephthalate (PETE). Pore size can vary from as small as 0.1 μm to as large as over 10 μm although the pore size should not be so large as to compromise the integrity of the conductive layer, preferably the pore size is from 0.1 μm to ≤10 μm. Membrane thickness can range from as thin as less than 5 μm to as thick as more than 100 μm, preferably 5 μm to 100 μm, more preferably between 5 μm and 25 μm.

In use, when submerged in fluid the porous membrane may permit the transfer of molecules, nutrients, reagents, gasses, cell metabolites etc. through the porous membrane, thereby creating perfusion cell culture conditions. In some embodiments, the porous membrane enables cell-to-cell communication and/or cell migration.

While certain geometries may be preferred for some embodiments, in its broadest embodiments, the membrane containing the electrodes can be of any geometry. One example geometry is rectangular, with a width between less than 1 cm to over 10 cm, in some embodiments between 1 cm and 10 cm, and a length between less than 2 cm to over 20 cm, in some embodiments between 2 cm and 20 cm. Other exemplary geometries are circular or any polygonal shape. As detailed further below, the electrode orientations and geometries may vary, with a single measurement electrode (which may also be referred to as a working electrode or sensing electrode or measurement electrode) and a single counter electrode (which may be referred to as a reference electrode) as a unit, or multiple measurement electrodes and single reference electrode as a unit, or multiple interdigitated electrodes as a unit, or multiple castellated electrodes as a unit. While in some embodiments, counter electrode and reference electrode may be used interchangeably, in other embodiments, the electrode may include both a counter electrode and a further reference electrode.

### Electrodes

In various embodiments, electrodes and associated structure units may have varying geometries and can be fabricated on the membrane by any suitable microfabrication method. Non-limiting examples of suitable fabrication methods are UV lithography, hot embossing, electron beam deposition and sputtering. Examples of electrode geometries are interdigitated electrodes, circular electrodes, rectangular electrodes, and castellated electrodes. Dimensions of the electrode feature geometries refer to the smallest width of an electrode feature, or the smallest distance between adjacent electrode features, and may vary from as small as less than 10 μm, to as large as over 1 mm. Electrode size can be as small as 125 μm diameter, and larger than 2 mm e.g. 3 mm; however, in a preferred embodiment, electrodes have a diameter of ≥250 μm to ≤1 mm.

The electrodes are suitably formed of gold or gold alloys.

As detailed in the Examples, the effect of electrode size on PM-ECIS measurements of endothelial cell barrier growth and disruption and the correlation between PM-ECIS vs. TEER resistance were assessed, and it was determined that PM-ECIS sensitivity to changes in barrier resistance and impedance depend on electrode size, with smaller electrodes being most sensitive to cell growth and barrier formation and disruption.

In some embodiments, the electrodes are prepared using a novel gold or alloy leaf process. This method can enable preparation of electrodes using materials not conducive to sputtering. Electrodes prepared according to these methods may be distinguished from electrodes prepared using sputtering methods. In particular, the deposited electrode components are continuous i.e. they extend across pores in the membrane, have a more consistent thickness as compared to those formed using sputtering and may be thinner than those produced by sputtering methods. These features are advantageously reflected in lower electrode resistance values, which allow for higher sensitivity measurements. In some embodiments, the electrode resistance value may be e.g. 10%, 20%, 30%, or 40% lower than the electrode resistance value of an electrode having the same configuration and components, but manufactured using a sputtering technique.

Also provided is a novel process (described in more detail below) of preparing electrodes using a porous sheet pre-coated with the electrically conductive material that employs a thermoplastic film, preferably Parafilm™, which is efficient and cost-effective compared to known preparation methods.

### Microfluidic PM-ECIS

In some embodiments, there is provided a novel PM-ECIS platform that combines microfluidic architecture to create a microenvironment suitable for modeling various tissues and tissue environments.

The electrode configurations provided herein enable sizing such that the (working and counter) electrodes can fit within a microfluidic channel. In some embodiments, the channels housing the electrodes have a width of between about 125 μm and 4 mm, preferably up to 3 mm, which it has been determined reduces the probability of bubbles occluding working electrodes.

In some embodiments, the present invention comprises a microfluidic device with an upper microchannel and a lower microchannel separated by a porous membrane (i.e. an ECIS membrane electrode). While references are made to an “upper” channel and a “lower” channel, it will be understood by a person of skill in the art that the device (and components thereof) may be maneuvered in space (particularly before seeding of cell cultures), such that an upper component may be below a lower component; in this sense, in some embodiments, these terms are not limiting, but are used to facilitate understanding of aspects of the inventive devices and methods.

The upper microchannel can have dimensions ranging 50 μm-5000 μm, preferably 125 μm-3000 μm in width, and 50 μm-3000 μm, preferably 120 μm-360 μm in height. Similarly, the lower microchannel can have dimensions ranging 50 μm-5000 μm, preferably 50 μm-3000 μm in width and 50 μm-3000, preferably 120 μm-360 μm in height. (The width and heights of the upper and lower channels may be the same or different.) The porous membrane can have thickness ranging from less than 5 μm to as thick as more than 100 μm, in some embodiments 5 μm to 100 μm. In preferred embodiments, the fabrication of the microchannels comprises laser cutting of double-sided tape that can be present in one layer or stacked in two or more layers to form the vertical walls of the microchannels. Some embodiments have more than two microchannels.

Each of the microchannels are suitably connected to circular ports that open up at the top surface of the device, allowing for influx (inlets) and efflux (outlets) of culture medium, assay medium, cell suspensions, or other fluids. The inlets can be connected via tubing to a pump that moves fluid from a reservoir into the device to generate laminar flow and fluid shear, and the outlets can be connected via tubing to receptacles to collect effluent for further analysis. The top and bottom surfaces of the device can be selected so as to facilitate imaging and, suitably, can have a thickness between about 0.15 mm and 10 mm, the thickness optionally being the same or different, e.g. to optimize imaging or support for tubing inserts.

In one embodiment, the top surface of the device is comprised of a transparent thermoplastic e.g. polycarbonate, polystyrene, cyclic olefin copolymer, that can be milled or laser cut, and range in thickness from less than 0.5 mm to˜10 mm. In one embodiment, the top surface of the device is formed of polymethyl methacrylate (PMMA) The bottom layer of the device is comprised of a solid transparent substrate (e.g. glass or PMMA), and can range in thickness from less than 0.15 mm to more than 5 mm, in some embodiments 0.15 mm to 5 mm. In another embodiment, the inlets and outlets are operatively connected to allow for recirculatory flow of culture medium, assay medium or other fluids. Another embodiment has fluid flow only through the upper microchannel, and a static culture in the lower microchannel, containing, in various non-limiting embodiments: a) static fluid, with cells grown on the underside of the membrane, b) static fluid with cells grown on the solid substrate, c) static fluid with cells grown both on the underside of the membrane and on the solid substrate, or d) cells suspended in a hydrogel that fills the lower microchannel.

Each device comprises at least two gold electrodes integrated onto the porous membrane on which cells are grown directly. In each device there is at least one counter electrode and at least one sensing electrode. Each electrode is connected by a thin, insulated gold trace integrated onto the membrane to a non-insulated connecting pad located on the outer edge of the device. The connecting pads are formed by an extension of the membrane with widened gold traces integrated into the membrane.

In some embodiments, the working electrodes are positioned along the length of a microchannel. In some embodiments, the working electrodes are congregated in a centre of the microchannel distal from the inlets and outlets, which can be beneficial for imaging and sensing due to lower turbulence.

The electrodes are connected to a device capable of producing alternating current signal, a device capable of measuring impedance signal output, and a computing device for controlling measurement timing and frequencies, as well as selection of devices to be measured. In a preferred embodiment, the functions of producing alternating current and measuring output signal are integrated into one piece of equipment, a lock-in amplifier.

### Manufacture of ECIS Membrane Electrodes and Microfluidic PM-ECIS Devices

While in some embodiments, electrode fabrication for novel microfluidic PM-ECIS devices and methods disclosed herein can be achieved using standard UV lithography techniques on gold-sputtered porous membranes, in preferred embodiments, the electrodes are fabricated according to the novel methods provided herein.

In various embodiments, electrodes can be shaped by a variety of methods, e.g. any form of photomask; electrode masking (insulating layer) made with packing tape, laser cut to shape to define exposed electrode.

In some embodiments, the electrodes are prepared using a novel gold or alloy leaf process. The bonding process relies on the porous membrane reaching its glass transition temperature and mechanically bonding to the gold (or gold alloy) leaf. By controlling the bonding temperature and pressure, bonding can be achieved without deformation or clogging of the pores in the membranes or compromising their integrity.

Heat-bonding pre-manufactured gold sheets or gold alloy sheets (e.g. copper, silver) directly to porous membranes has numerous advantages over traditional metal deposition protocols. In particular, heat-bonding gold (or alloy) leaf provides a flat continuous substrate that permits standard large scale photolithography techniques and results in improved conductivity over deposition methods. For example, the inventors measured electrode resistivity of commercial roll-to-roll sputter-coated devices to be 6.6×10⁻⁸±1.1×10⁻⁸Ω·m, whereas the resistivity of the same electrode pattern fabricated by gold leaf heat-bonding was significantly lower at 5.2×10⁻⁸±1.5×10⁻⁸Ω·m (p=0.015); lower electrode resistance values are more desirable as they allow for higher sensitivity measurements. The leaf heat-bonding method also allows for the use of biocompatible gold alloys (silver and copper) that further improve electrical conductivity and electrode integrity. Finally, there is minimal material wastage with leaf heat-bonding, as the only gold lost during fabrication is the negative space between the electrodes. In evaporating or sputtering methods, the majority of the gold is wasted due to the low solid angle ratio between the exposed substrate and the rest of the machine. It is estimated that for the PM-ECIS devices disclosed herein, fabrication by standard thermal evaporation and photolithography techniques would be ˜4-fold more expensive and take ˜35% longer than gold leaf bonding.

In one example embodiment, electrode fabrication comprises:
a) hot embossing gold or gold alloy leaf and a porous membrane to a plate, suitably a steel or glass plate, to provide a rigid substrate for photolithography;
   In some embodiments, the hot embossing step comprises:
a1) embossing a stack made up of the plate, the conductive sheet, which will typically be provided on a transfer paper, and a non-stick polymer sheet under conditions (time, temperature and pressure) that allow for initial bonding while permitting removal of the transfer paper once the stack is cooled;
   For example, in one embodiment, step a1) can include embossing a stack of (from bottom to top): the plate (e.g. a flat glass sheet), the porous membrane (e.g. PET porous track etched membrane) and the conductive sheet (e.g. gold leaf on transfer paper); and a non-stick Teflon sheet in a hot embosser for 10 minutes at 2.3 MPa and 180° C.
   The optimal conditions will depend on the plate, porous membrane and conductive sheet used (material and thickness). In some embodiments, these conditions may fall within the ranges 10 minutes-20 minutes at 2 MPa-5 MPa and 180° C.-190° C.
   Suitably a conductive sheet, preferably a gold or a gold alloy leaf, having a thickness of between about 200 nm and 500 nm is used, in some embodiments a sheet having a thickness of 250 nm±10.
a2) cooling the stack, preferably to room temperature under pressure, and then removing the gold leaf transfer paper;
   While in some embodiments, the stack may be allowed to cool passively, in other embodiments, the stack may be actively cooled. The stack is suitably cooled to a temperature that enables removal of the transfer paper without substantial residue. In some embodiments, wherein active cooling is employed, the rate of cooling is suitably ≤6° C. per minute.
a3) hot embossing the stack (minus the transfer paper) to complete the bonding process;
   In this context, completing the bonding process refers to bonding so as to enable a smooth conductive surface, which is fully bonded to the membrane i.e. remains bonded to the membrane for the purpose of subsequent photolithographic steps and during use of the assembled device.
   For example, in one embodiment, the plate (e.g. a flat glass sheet), the porous membrane (e.g. PET porous membrane) and the conductive sheet (e.g. gold leaf); and the non-stick sheet are embossed in a hot embosser for 10 minutes at 9.2 MPa and 200° C.
   The optimal conditions will depend on the plate, porous membrane and conductive sheet used (material and thickness). In some embodiments, these conditions may fall within the ranges 10 minutes-20 minutes at 6.5 MPa-9.5 MPa and 200° C. to 210° C.
a4) cooling the stack, preferably to room temperature under pressure.
   While in some embodiments, the stack may be allowed to cool passively, in other embodiments, the stack may be actively cooled. In some embodiments, wherein active cooling is employed, the rate of cooling is suitably ≤6° C. per minute.
   The initial thermal bonding step allows for easy removal of gold leaf transfer paper, whereas the second bonding step fully bonds the membrane to the gold, leaving a smooth gold surface.
   The hot embossing step is followed by forming electrode components using photolithography techniques, which generally include applying a photoresist over the conductive surface; placing a photomask over the photoresist, exposing the photoresist to UV light, removing unexposed photoresist, and using an etching solution to remove conductive material not covered by the exposed photoresist. And, in a specific example embodiment, includes:
b) applying a dry film photoresist over the gold- or gold alloy-coated membrane using a laminator at 20-25° C.;
c) placing a photomask on top of the photoresist, and exposing the photoresist to a UV lamp for 8-10 seconds;
d) removing unexposed photoresist, suitably using 1% sodium carbonate in water solution, and washing, suitably with isopropanol, to remove any residue;
e) using gold etch, suitably TFA gold etch (suitably, gold etch time can vary from 3-8 min, with or without agitation), to remove gold/gold alloy not covered by the exposed photoresist, and washing, suitably with acetone, to remove and traces of etching solution; and
optionally, f) removing the remainder of the photoresist, suitably using 0.5 M NaOH (alternatively, the wash of step e) can be extended for this purpose).
   This method is exemplified in Example 1.

In another embodiment, electrode fabrication employs a “pre-coated” membrane, in one embodiment, a pre-coated PETE membrane, coated with sputtered gold. In one example, this fabrication method includes:
a′) Laminating a thermoplastic film to a substrate, preferably a flexible substrate.
   Suitably, the thermoplastic film can be rendered adhesive through the application of pressure and heat. The thermoplastic suitably comprises a blend of waxes and polyolefins. In one embodiment, the method employs the thermoplastic film Parafilm™ (Parafilm M All-Purpose Laboratory Film, Amcor). In an example embodiment, Parafilm™ is laminated to a PETE sheet having a thickness of between about 100 μm and 800 μm, at a pressure ranging between 2 kg/cm² and 5 kg/cm², at a temperature of 20-25° C.
   In various embodiments, the flexible substrate may be any thin (in some embodiments 100 μm to 800 μm in thickness) substrate sheet compatible with the thermoplastic film so as to enable lamination. A flexible substrate is preferred, as in some embodiments, both the thermoplastic film and substrate may be provided as a roll, enabling efficient and simplified manufacturing of the electrode. Examples of suitable substrate include polycarbonates. In one embodiment, the substrate is a PET sheet.
b′) Laminating porous membrane pre-coated in sputtered gold to the thermoplastic film layer of the laminate of step (a′), suitably using the same pressure and temperature settings as in step a′).
   In some embodiments, a′) and b′) may be performed concurrently.
c′) Applying heat and pressure to the laminate of step b′) such that the porous membrane and thermoplastic film adhere through steps d′) to k′), but are only adhered to such an extent that post-ECIS manufacture, the porous membrane can be removed from the thermoplastic-substrate layer. In one embodiment, hot embossing the laminate, in an example embodiment, a hot embosser may be used to apply a pressure between 2 kg/cm² and 5 kg/cm² at a temperature of 54-56° C. for 1 min. While step c′) may be a separate step, in an alternative embodiment, the heat and pressure settings of c′) may be applied as part of the lamination process.
   Followed by forming electrode components using photolithography techniques, which generally include applying a photoresist over the conductive surface; placing a photomask over the photoresist, exposing the photoresist to UV light, removing unexposed photoresist, and using an etching solution to remove conductive material not covered by the exposed photoresist. And, in a specific example embodiment, includes:
d′) Laminating dry film photoresist to membrane, suitably using same pressure and temperature settings as in step a′).
   While step d′) may be a separate step, in an alternative embodiment, step d′) may be performed concurrently with step c′), or steps a′) through d′) may be performed concurrently. Such embodiments may have particular application when the method is implemented as a roll-to-roll process, which may be automated, enabling efficient electrode fabrication.
e′) Placing photomask on top of dry film photoresist, and exposing to UV lamp for 8-10 seconds.
f′) Baking in a 60° C.-80° C., preferably a 70° C. oven for 5 min.
g′) Using 1% sodium carbonate in water solution, washing away unexposed photoresist.
h′) Washing with isopropanol to remove photoresist residue.
i′) Using TFA gold etch solution to remove gold not covered by exposed photoresist. Suitably, gold etch time can vary from 3-8 min, with or without agitation.
j′) Washing with acetone to remove traces of gold etch solution.
optionally, k′) Using 0.5 M NaOH in water solution, remove remaining photoresist. (Alternatively, the wash of step j′) can be extended for this purpose)

Following either fabrication method, the ECIS membrane electrode can be peeled off the substrate without (or substantially without) residue and cut to size using a laser cutter. A stream of running water aimed at the junction of membrane and the substrate (e.g. steel plate) may facilitate separation. In an alternative embodiment, the product of the second fabrication method may be cut, e.g. cut with a blade (manually, Cricut™) before the membrane is removed from the substrate.

Advantageously, both methods are cost-effective and simple, allowing for start-to-finish fabrication and assembly that does not require cleanroom facilities and enable rapid prototyping.

Once formed, the ECIS membrane electrode may be used to fabricate a PM-ECIS device, in some embodiments, a PM-ECIS device as exemplified herein, in some embodiments a microfluidic PM-ECIS device as provided herein.

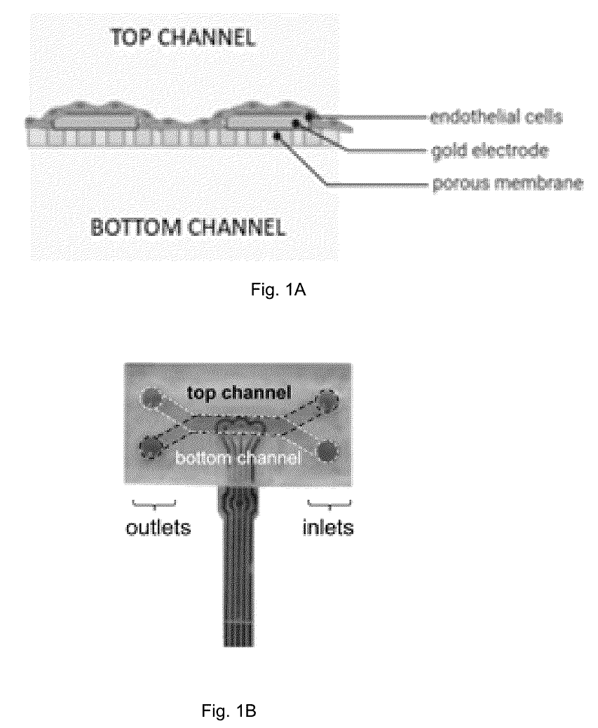

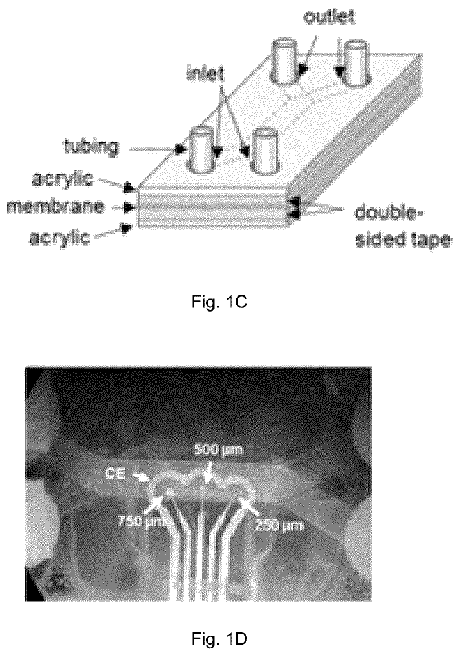

In one example, the membrane may be suspended between top and bottom channels (formed on upper and lower substrates or plates e.g. acrylic plates) by sandwiching with double-sided tape (per e.g. FIG. 1C). As mentioned above, the fabrication of the microchannels can be by way of laser cutting of double-sided tape. that can be present in one layer or stacked in two or more layers to form the vertical walls of microchannels. Any optically transparent, biocompatible double sided tape may be suitable, e.g. ΔRseal™ 90880; ΔRcare™ 90106NB. The use of double-sided tape as an affixing method advantageously can minimize handling and damage to the membrane electrode (vs. other methods of positioning the membrane into microfluidic channel (solvent bonding, hot embossing, liquid adhesives etc.))

The electrodes, via connecting pads, are linked to a device capable of producing alternating current signal, a device capable of measuring impedance signal output, and a computing device for controlling measurement timing and frequencies, as well as selection of devices to be measured. In a preferred embodiment, the functions of producing alternating current and measuring output signal are integrated into one piece of equipment—a lock-in amplifier.

In one embodiment, the connecting pads are inserted into a flexible flat cable connector integrated onto a flexible printed circuit board (PCB) that can be inserted into a 6-well plate. For a microfluidic device such as shown in FIG. 1B, distance between device and electrical point of connection to the flexible printed circuit board was increased to accommodate possible leaking/make additional space for tubing. The flexible PCB is in turn connected in some embodiments to a circuit board that integrates 24-channel multiplexing capabilities with a commercially available microcontroller chip to allow for interfacing with a lock-in amplifier, computer and custom application that allows for real-time visualization of data.

In another embodiment, the devices are placed in a device station—designed for housing cell culture inserts in a well plate or for securing microfluidic devices—with at least two connector pins coming in contact with the at least two connection pads on each device. The connector pins can be similarly connected via circuitry to a device capable of producing alternating current signal, a device capable of measuring impedance signal output, and a computing device for controlling measurement timing and frequencies, as well as selection of devices to be measured.

### Culturing Cells

The methods and devices described herein may employ cell seeding techniques known to those of skill in the art. Examples are provided herein. In some embodiments, cell seeding density may be increased to accommodate for microfluidic proportions.

The present invention enables culturing of cells in a microfluidic device and simultaneously monitoring cells that are cultured on the electrodes. In some embodiments, the microchannels can be loaded/contain, in working embodiments, about 70 nL to about 810 μL, preferably about 420 nL to about 30 μL culture medium. (The upper and lower microchannels may contain different volumes and the inlets and outlets may contain further volumes.) Following fabrication, devices are suitably twice rinsed with 500 μL of 70% ethanol in water solution, then rinsed with 1 mL distilled water. A vacuum line may be used to remove any residual liquid. The devices are then placed in a plasma cleaner and treated with oxygen plasma for 3 min, followed by 1 hour UV sterilization in a biosafety cabinet including 30 minutes sterilization by filling channels with 70% ethanol solution. In some embodiments, the device is degassed immediately prior to use to reduce the probability of bubbles occluding working electrodes. Following a rinse with 500 μL phosphate buffer saline solution, the microchannels can be coated with protein solution, filling the channel to ensure that all the surfaces are in contact with the solution. To stabilize the impedance readings, the devices are filled with the intended culture medium, placed in an incubator at 37° C., 5% CO₂ and impedance measurements taken for at least 6 hours and up to more than 24 hours to obtain baseline values. Then cell suspensions can be added and impedance measurements can be taken immediately thereafter. While appropriate frequencies will vary by cell type and application, a range of 10 kHz to 60 kHz can be used to monitor cell coverage of electrodes over time, and a range of 1 kHz to 10 kHz can be used to monitor barrier integrity. Timing and amount of shear applied for fluid flow is determined by application.

Suitably, the cell culture medium is also degassed immediately prior to use, which may be done according to degassing methods known in the art. In some embodiments, degassing of the culture medium may be performed using a vacuum chamber, membrane degasification or vacuum filtration. In one embodiment, the cell culture medium may be allowed to degas by being stored for a suitable period of time (e.g. overnight) in a vessel with a gas permeable cap, suitably in an incubator.

### Application of the PM-ECIS Devices

The devices provided herein can enable for direct, non-invasive, and real-time assessment of cells cultured on porous membranes.

The devices as provided herein may be used, for example, to measure one or more of: cell proliferation, cell spreading, endothelial or epithelial barrier function, cell ion channel activities, cell ligand binding and signaling, cell metabolism, cell cytotoxicity, adhesion of circulating cells to the endothelium or epithelium, transendothelial or transepithelial migration of cells (such as immune cells, metastatic cells, circulating stem cells, pathogens, etc.), and transport of drugs and drug delivery vehicles across the endothelium or epithelium.

In one embodiment, methods provided herein may comprise a step of imaging cells in the microfluidic device. For example, in embodiments wherein the device comprises an upper supporting plate having defined thereon an upper cell-culture channel and a lower supporting plate having defined thereon a lower cell-culture channel operable to hold a cell-culture medium, one or both channels of the device may be imaged from below or above through a supporting plate made of a suitable material using imaging equipment known in the art (e.g., a microplate reader). Advantageously, cells subjected to a flow based assay using the microfluidic device provided herein may be imaged at one or more time points during the assay without requiring draining of the fluid from the device or disassembly of the device.

In one embodiment, a method for culturing cells in the microfluidic device is provided. Cells or tissues may be cultured encapsulated in a hydrogel, other matrix, unencapsulated or on a scaffold. Any cell or tissue of interest may be cultured. For example a cell may be normal, mutant, cancerous or diseased. The cell may be derived from any unicellular organism (e.g., bacteria, protists) or multicellular organism (e.g., animal, plant, etc.). The tissue may be derived from any multicellular organism. The cultured cells or tissues may be a single cell or tissue type or a plurality of cell and/or tissue types. One or more cell or tissue types may be cultured simultaneously in the microfluidic device. A single cell or tissue type may be cultured separately in separate culture chamber wells, or more than one cell or tissue type may be cultured in a chamber.

In one embodiment, different types of cells or tissues representative of the body (e.g., human or mammalian) may be cultured, e.g., heart, kidney, liver, lung, heart, stomach, intestines, brain, neurons, glia, pancreas, ovary, muscle (skeletal, cardiac, smooth, etc.), skin, etc. Multiple cell or tissue types may be cultured in 2D or 3D configurations of the microfluidic device provided herein under flow or static (i.e., non-flow) conditions.

Notably, the electrode dimensions and fabrication method for PM-ECIS make it compatible not only with conventional co-culture setups, but also with microfluidic organ-on-a-chip configurations. Without limiting the generality of the foregoing, the methods and devices provided herein may have application in any organ-on-a-chip model that has an endothelial or epithelial monolayer component.

It is contemplated that various model systems may be developed using one or more embodiments of the device provided herein. For example, various endothelial and/or epithelial tissue systems may be modeled using one or more embodiment of the devices provided herein, such as, but not limited to: vascular systems (i.e, tissue systems comprising an endothelial-vascular smooth muscle cell interface); valvular systems (i.e, tissue systems comprising an endothelial-valvular interstitial cell interface); cardiac systems (i.e, tissue systems comprising an endocardial-cardiomyocyte-fibroblast interface); gut systems (i.e, tissue systems comprising an intestinal epithelial-stromal cell interface); ocular systems (i.e, systems comprising a retinal epithelial-endothelial interface); cancer systems (i.e, tissue systems comprising metastatic cells and an endothelial parenchymal tissue interface); or immunology systems (i.e, tissue systems comprising a blood cell-endothelial cell interface).

In various embodiments, the devices provided herein may be used to model extravasation and transport from the blood to the subendothelial space such as, for example, immune cell trafficking, metastasis, transport of drugs or other compounds, nanoparticle transport, etc.

In one embodiment, the devices provided herein may be used for performing studies of the effects of drugs, toxins or other chemical agents on the cultured cells. For example, testing of the toxicity of chemical compositions, drugs and other compounds of interest may be examined using the microfluidic device provided herein.

Some non-limiting examples of such model systems are provided below.

### Blood-Brain-Barrier (BBB) Model System

In various embodiments, a porous membrane comprising electrodes suitable for culture of cells thereon is provided in a device suitable for BBB modeling.

For example, in one embodiment, brain microvascular endothelial cells are seeded in a microchannel in a first cell culture layer, for example, on an apical surface of the porous membrane on which the electrodes are patterned. Suitably, this surface is coated with one or more extra cellular matrix proteins, such as fibronectin. Astrocytes may be seeded on a basal surface of the porous membrane. In some embodiments, natural (e.g., collagen I) or synthetic (e.g., polyethylene glycol) hydrogels embedded with astrocytes may be polymerized directly inside the second cell culture layer. Optionally, other cells of interest (e.g., neurons and/or other glial cells) may be added to one or more of the culture channels. When the seeded first and second cell culture layers are co-cultured, vascularized brain microtissues can be generated. The generated vascularized brain microtissue comprises endothelium, which a user can subject to physiological shear stresses by flowing media through the device. In one embodiment, primary human cells are seeded into the cell culture layers. In this embodiment, a human-like blood-brain-barrier can be generated. Such a system may, for example, be suitable for screening drug candidates and/or drug delivery vehicles. Such a system may, for example, be suitable for investigating BBB-related biological mechanisms.

### Cell Perfusion Model System

In another example, in one embodiment, the device provided herein is used to assess and/or monitor fluid and/or nutrient exchange in a cell perfusion system. The measurements obtained using the inventive devices and methods, may be used as indicators for timing of medium replenishment based on output values reflecting cell coverage and barrier integrity.

In one embodiment, a perfusion model system comprising cells cultured in a device provided herein may improve cell growth and/or health by exchanging the cell culture medium continuously, thereby delivering fresh nutrients and washing away waste products continuously in contrast to standard tissue culture in which the medium is changed periodically (e.g., every other day). For example, a user may set up a cell culture in the device provided herein to perfuse for a period of time, spent medium being exchanged for fresh medium periodically.

### Example 1: Membrane Electrode and Device Fabrication

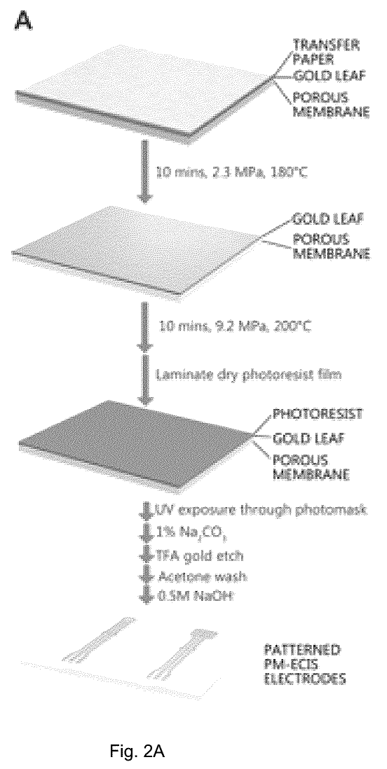

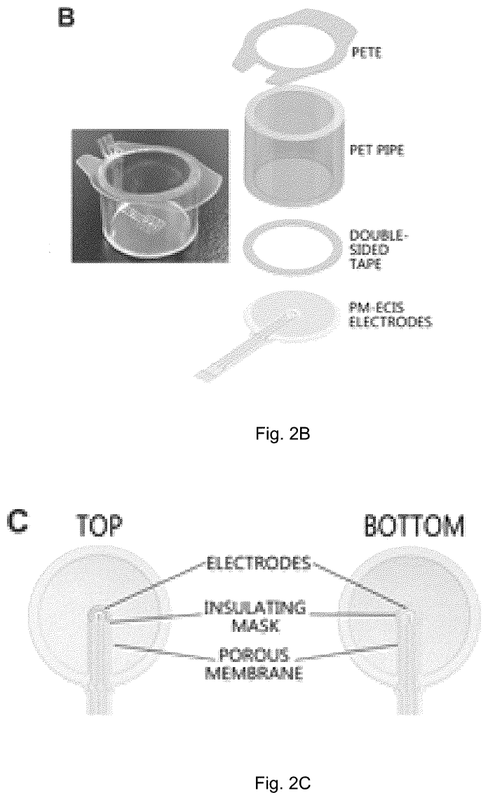

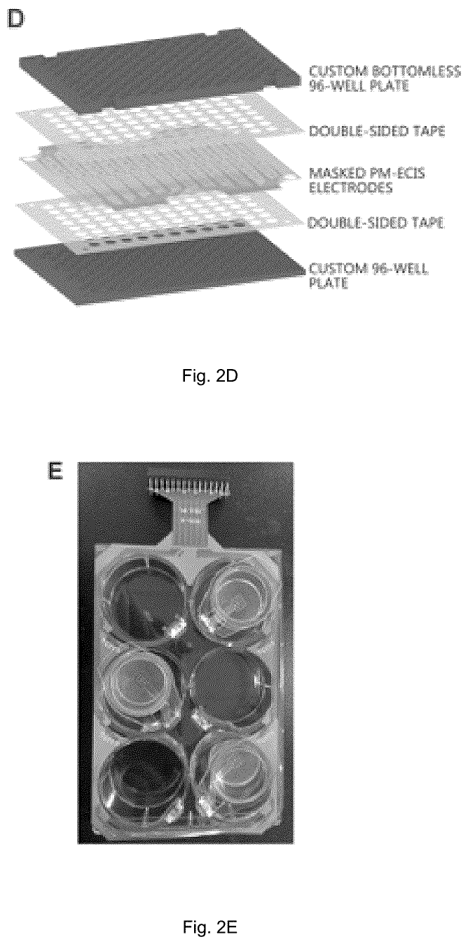

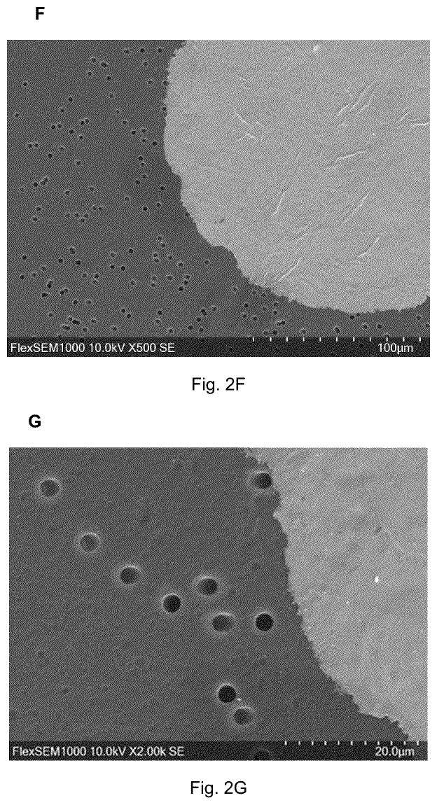

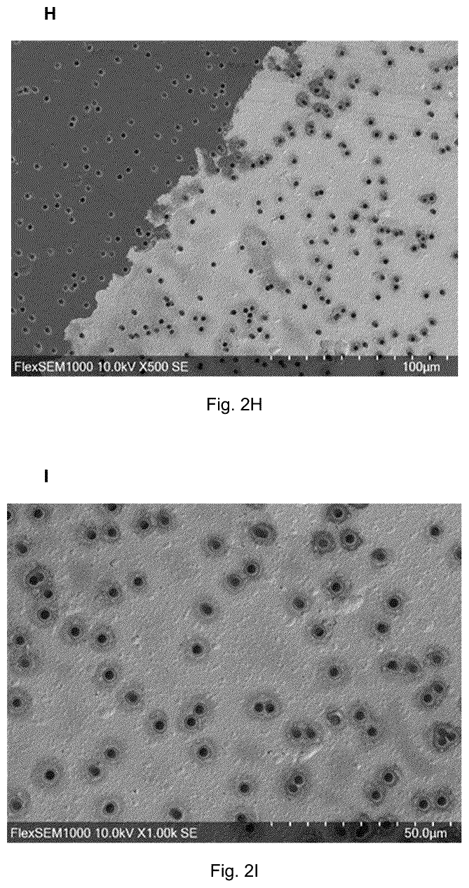

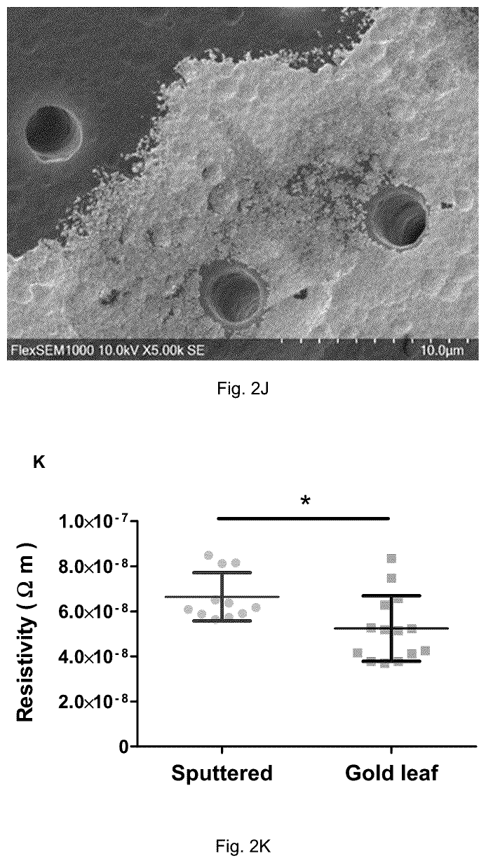

With reference to FIG. 2A, porous culture membranes were patterned with electrodes by heat-bonding gold leaf to the membranes. Briefly, a stack consisting of (bottom to top): a flat glass sheet; polyethylene terephthalate (PET) porous track etched membrane (Sterlitech); a 200 nm thick gold leaf (on transfer paper; Gold Leaf Supplies); and a non-stick Teflon sheet (8569K34, McMaster-Carr), were bonded in a hot embosser (AutoSeries-3889, Carver) for 10 minutes at 2.3 MPa and 180° C. After letting the stack cool to room temperature under pressure, the gold leaf transfer paper was removed, and the stack placed back into the hot embosser to continue the bonding process for another 10 minutes at 9.2 MPa and 200° C. Finally, the stack was left under pressure to cool to room temperature. The initial thermal bonding step allowed for easy removal of gold leaf transfer paper, whereas the second bonding step fully bonded the membrane to the gold, leaving a smooth gold surface. A sheet of dry film photoresist (MonkeyJack) was then bonded to the gold surface using a laminator (ASC365, Thermal Laminating) at room temperature. Using a photomask, the negative pattern was exposed to UV for 10 seconds (OmniCure S2000, Lumen Dynamics) and developed in 1% Na₂CO₃ solution. The gold was etched using Gold Etchant TFA (Transene Company Inc. Danvers, MA) for 8 minutes (visually inspected to ensure all unwanted gold was removed) and washed with distilled water. The substrate was briefly washed with acetone to remove only the residual TFA etchant and then rinsed with distilled water. The remaining photoresist was removed using 0.5 M NaOH, after which the substrate was washed and left to soak in distilled water for at least 2 hours to ensure delamination of the membrane off the glass.

For cell culture, patterned PM-ECIS electrodes were cut using a laser cutter (VLS 3.5, Universal Laser Systems) for integration into custom-made 6-well inserts and 96-well devices. Inserts for 6-well plates were fabricated using polyethylene terephthalate glycol (PETG) pipe (McMaster-Carr, #9245K37) with outer diameter=1″, inner diameter=7/8″ cut to a height of 17.5 mm using a pipe cutter. A hot embossing step (140 kPa, 70° C. 3 min.) was used to create a smooth surface at the cut sites to facilitate subsequent assembly steps. Top frames for the inserts were created from laser cut polyester sheet (McMaster-Carr, #8567K92), and solvent bonded to the PETG pipe segments using SCIGRIP 4 Acrylic Cement (McMaster-Carr, #7517A2). A second hot embossing step (140 kPa, 70° C. 3 min.) further enhanced bonding between the polyester sheet and PETG pipe components. Finally, double-sided tape (ΔRSeal 90880, Adhesives Research) was used to secure PM-ECIS electrodes to the bottom face of the inserts; FIG. 2B summarizes assembly of the 6-well inserts.

Electrical insulating masks were laser cut from clear tape (Grand & Toy, #99842), aligned, and bonded on both sides of the membrane through cold lamination followed by hot embossing (2.15 kPa, 60° C., 2 min) to cover gold traces and restrict the sizes of electrodes (FIG. 2C).

PM-ECIS electrodes for 96-well devices were fabricated similarly to those in the insert embodiment. The electrodes were assembled with layers of laser cut double-sided tape and custom micro-milled polycarbonate 96-well plates (FIG. 2D).

Flat flex cable (FFC) gold finger connectors were added to the PM-ECIS electrodes to interface with a flex printed circuit board that connected the devices to the ECIS measurement system via an Arduino-operated multiplexer, allowing for measurements of up to 24 individual electrodes in parallel (FIG. 2E).

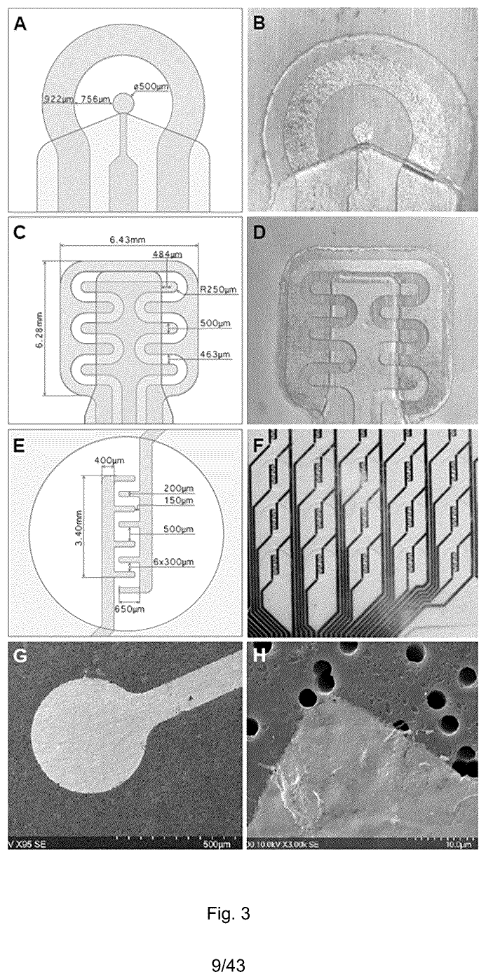

Three electrode configurations were tested. For the 6-well plate inserts, either a single 500 μm diameter electrode (FIG. 3A,B; surface area 0.19635 mm²) or a six-finger electrode (FIG. 3C,D; total surface area of 6×0.340175 mm²=2.04 mm²) was used. For the for 96-well plate, eight interdigitated electrodes were patterned (FIG. 3E,F; total electrode surface area of 4.933 mm²)

Fabrication methods such as metal evaporation (thermal and e-beam) and sputtering are the current industry-standard methods for depositing gold electrodes onto non-conductive surfaces due to their versatility in a wide range of applications. They are limited, however, by high material and time costs that make post-prototyping manufacture challenging. The gold leaf transfer protocol exemplified here and shown schematically in FIG. 2A is a simple, cost-efficient alternative to metal deposition for manufacturing membrane electrodes for PM-ECIS. The bonding process relies on the membrane reaching its glass transition temperature and mechanically bonding to the gold leaf. By controlling the bonding temperature and pressure, bonding can be achieved without deformation or clogging of the pores in the PET membranes or compromising their integrity (see Example 2).

FIGS. 2F through J compare gold leaf hot embossed as employed in the process shown in FIG. 2A vs. e-beam deposited gold onto porous membrane. Hot embossed gold leaf forms a continuous metal layer that spans the pores in the membrane, which is left intact after UV lithography using TFA gold etch solution, while applying the same etching process to an e-beam deposited gold membrane yields a non-uniform metal layer with heterogeneous coverage of the membrane and discontinuities at the pores.

Heat-bonding pre-manufactured gold sheets directly to porous membranes has numerous advantages over traditional metal deposition protocols. In particular, heat-bonding gold leaf provides a flat continuous substrate that permits standard large scale photolithography techniques and results in improved conductivity over deposition methods. For example, FIG. 2K compares electrode resistivity measured for commercial roll-to-roll sputter-coated devices to be 6.6×10⁻⁸±1.1×10⁻⁸Ω·m, whereas the resistivity of the same electrode pattern fabricated by gold leaf heat-bonding was significantly lower at 5.2×10⁻⁸±1.5×10⁻⁸Ω·m (p=0.015); lower electrode resistance values are more desirable as they allow for higher sensitivity measurements. There is minimal material wastage with leaf heat-bonding, as the only gold lost during fabrication is the negative space between the electrodes. In evaporating or sputtering methods, the majority of the gold is wasted due to the low solid angle ratio between the exposed substrate and the rest of the machine. For the PM-ECIS devices reported here, fabrication by standard thermal evaporation and photolithography techniques are estimated to be ˜4-fold more expensive and take ˜35% longer than gold leaf bonding as described. The leaf heat-bonding method also allows for the use of biocompatible gold alloys (silver and copper) that further improve electrical conductivity and electrode integrity.

### Example 2: Cell Culture and Characterization Using PM-ECIS Devices

### Cell Culture

Cells were cultured at 37° C. and 5% CO₂ in cell-specific media: primary human umbilical vascular endothelial cells (HUVECs, Lonza; up to passage 6) in EGM-2 (Lonza) culture medium kit; primary human brain microvascular endothelial cells (HBMVECs, Cell Systems—ACBRI 376) in complete classic medium (4Z0-500, Cell Systems) supplemented with CultureBoost™ (Cell Systems); immortalised HBMVECs (Boczula et al., 2021) in EGM-2MV microvascular endothelial cell growth medium (Lonza) supplemented with hygromycin-B (20 μg/mL); hCMEC/D3 BBB endothelial line (CELLutions Biosystems, Inc CLU512) cultured according to supplier's directions in supplemented EBM-2 endothelial basal medium (Lonza); and normal human astrocytes (NHAs, Lonza) in Dulbecco's minimum essential medium (DMEM), supplemented with N2 supplement (1%) and fetal bovine serum (10%). Media was changed every 48 hours in cell culture flasks, and every 24 to 48 hours in PM-ECIS devices.

To prepare PM-ECIS devices for cell culture, devices were plasma treated (PDC-001-HP, Harrick Plasma) for 3 minutes and UV sterilised for 2 hours (on both sides). Fibronectin (100 μg/mL) was then added to the apical side (with electrodes), and Geltrex™ (100 μg/mL) to the basolateral side for devices used in co-culture experiments, for 30 mins at RT to promote subsequent cell adhesion. HUVECs were seeded at 100,000 cells/cm² or 250,000 cells/cm² as indicated; primary HBMVECs were seeded at 5,000 and 30,000 cells/cm² for permeability assays; and immortalized HBMVECs were seeded at 50,000 cells/cm². For co-culture, astrocytes were seeded on the basolateral side of the ECIS device membrane at 50,000 cells/cm² one day prior to endothelial cell seeding.

### Cell Staining

For immunostaining, cells were fixed in methanol for 10 min at −20° C., washed three times with PBS, and blocked with 3% BSA (Sigma-Aldrich, 10735086001) for 20 min at 37° C. Cells were stained with primary antibodies (1:100) diluted in 3% bovine serum albumin in PBS for 1 hour at 37° C. Following incubation, cells were washed three times with PBS and blocked using 10% goat serum (Sigma-Aldrich, G9023) in PBS for 1 hour at room temperature. Secondary antibodies (1:200) diluted in 10% goat serum were applied for 1 hour at room temperature. After washing three times with PBS, nuclei were stained with 1:1000 Hoechst 33342 (Sigma-Aldrich, B2261) for 5 min at room temperature. Primary antibodies were mouse monoclonal ZO-1 (Thermo Fisher Scientific, 33-9100) and rabbit polyclonal GFAP (ab7260; Abcam); goat anti-mouse IgG (H+L) Alexa Fluor 488 (A32723, Thermo Fisher Scientific) and goat-anti rabbit IgG (H+L) Alexa Fluor 568 (A11036, Thermo Fisher Scientific). For viability staining, cells were stained using a Live/Dead kit (Invitrogen, USA, cat #L3224) according to the supplier's protocol. Image acquisition was performed using an Olympus FV3000 confocal laser scanning microscope or Olympus BX51 inverted fluorescence microscope as indicated.

### PM-ECIS Devices Support and Monitor Endothelial Cell Adhesion, Growth, and Monolayer Barrier Formation

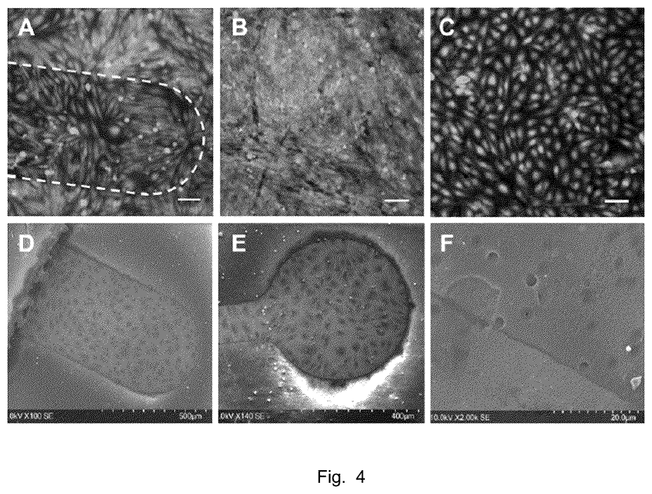

To test their biocompatibility, PM-ECIS devices were seeded with HUVECs. In 96-well PM-ECIS devices, HUVECs grew on the fibronectin-coated gold electrodes, tape masks, and PET membranes at similar densities and with similar viabilities and morphologies to HUVECs on standard tissue culture-treated polystyrene well plates (FIG. 4A-C). (Cell-free devices were imaged by scanning electron microscopy (Hitachi S-2500). To prepare for imaging, devices with cells were fixed with 4% paraformaldehyde for 2 hours and gradually dehydrated in 30%, 50%, 70%, 95% and 100% ethanol with 30 min incubation period for each step. The substrates were then sputtered with platinum (Polaron SC515 SEM coating system) and imaged.) SEM imaging further confirmed HUVEC attachment to electrodes of different shapes (FIG. 4D,E), with cells spanning the interface between the electrodes and the bare membrane (FIG. 4D-F). Moreover, SEM imaging confirmed the electrodes stayed fully adhered to membranes after at least 4 days in culture (FIG. 4D-F).

### ECIS Data Collection

Electrical impedance was measured at 400 Hz, 4 kHz, and 40 kHz in 96-well devices over 4 days of culture, with cells initially seeded at 100,000 or 250,000 cells/cm².

A lock-in amplifier (LIA) (SR850, Stanford Research Systems) was used to simultaneously generate AC current and read impedance data from the devices. An Arduino-operated multiplexer was used to switch between devices. Using a 1 V rms input voltage, in series with a 1 MΩ resistor, electrodes (typ. 500 μm dia.) were stimulated at frequencies of 400, 4000 and 40000 Hz, and any voltage drops (at the locked frequencies) across the electrodes were sampled with the LIA every 15 or 60 mins. Prior to use, the devices without cells were left in media for at least 12 hours to record a stable cell-free impedance baseline for each electrode at each frequency.

### Impedance Data Analysis

Parasitic impedance offset at t=0 was removed from all measurements, and then normalised to their effective exposed electrode areas (measured optically using a SZ61 Olympus stereoscope) using the following inverse sum equation:

$$\frac{1}{{A}_{effective}} = \frac{1}{{A}_{electrode}} + \frac{1}{{A}_{reference}} \qquad ( 1 )$$

where Aelectrode and the Areference are the areas of the two electrodes used for impedance measurement.

Cell barrier resistance (Rb), capacitance (Cm), and basal barrier beneath the cells (a) were modeled and calculated using ECIS Core software (Applied Biophysics, v1.2.215.0 PC) using the raw impedance values taken at 400, 4000, and 40000 Hz.

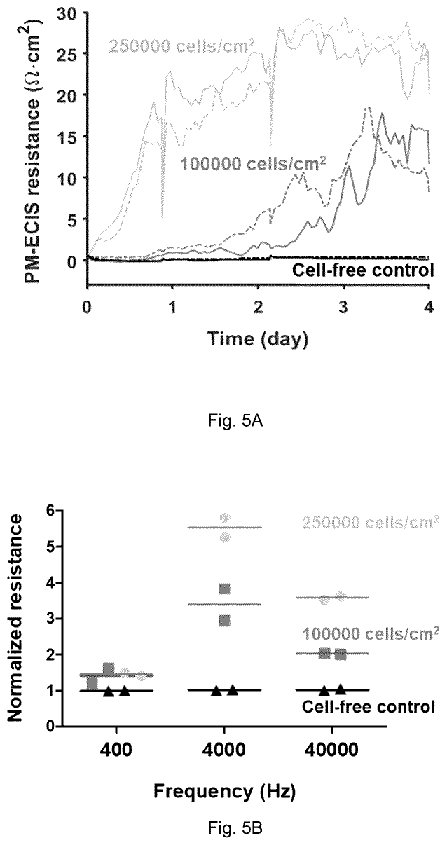

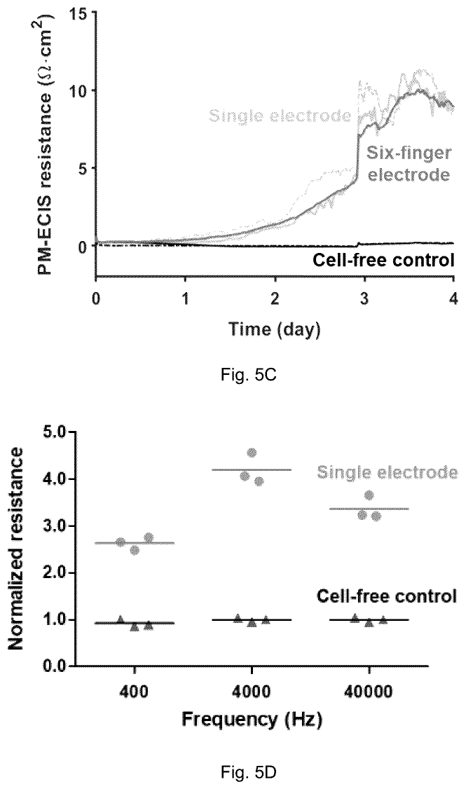

Resistance at 4 kHz increased over the culture duration, indicating increasing cell coverage of the electrodes (putatively due to spreading and proliferation) and increasing barrier function (FIG. 5A). As expected, cells seeded at 250,000 cells/cm² had greater resistance initially than cells seeded at 100,000 cells/cm² (FIG. 5A). Resistance in the higher density cultures also plateaued by day ˜2.5, suggesting confluence and barrier formation, whereas resistance of the lower density cultures continued to increase throughout the four days (FIG. 5A). Cell-free controls showed no change in resistance over the culture period. Similar to traditional solid substrate ECIS systems (Applied Biophysics, n.d.; Gamal et al., 2018), events in cell culture, including cell micromotion (high frequency oscillations in resistance) and media changes (abrupt changes in resistance), were also observed in the electrode signal (FIG. 5A).

The final cell monolayer resistances at 4 kHz were 10-30 Ω·cm2 (FIG. 5A,B), consistent with values from commercial solid substrate ECIS systems. Also similar to trends observed with commercial solid substrate ECIS systems cell monolayer resistance was greater at 4 kHz than 400 Hz or 40 kHz and was greater in the higher density cultures (FIG. 5B). HUVECs grown on the 6-well PM-ECIS inserts displayed a similar behavior to cells grown in 96-well plate devices. The resistance of cells seeded at 100,000 cells/cm² gradually increased over time in culture to ˜10 Ω·cm² by day 4, whereas cell-free controls showed no change (FIG. 5C). Furthermore, similar trends were observed for the two electrode designs tested in these experiments, although the six-finger electrode with larger area produced a steadier ECIS signal than the single 500 μm diameter electrode (FIG. 5C); this was similar to commercial ECIS devices with comparable electrode designs. Cell monolayer resistance peaked at 4 kHz for both electrode designs (FIG. 5D).

### PM-ECIS Devices Support and Monitor Cell Co-Culture

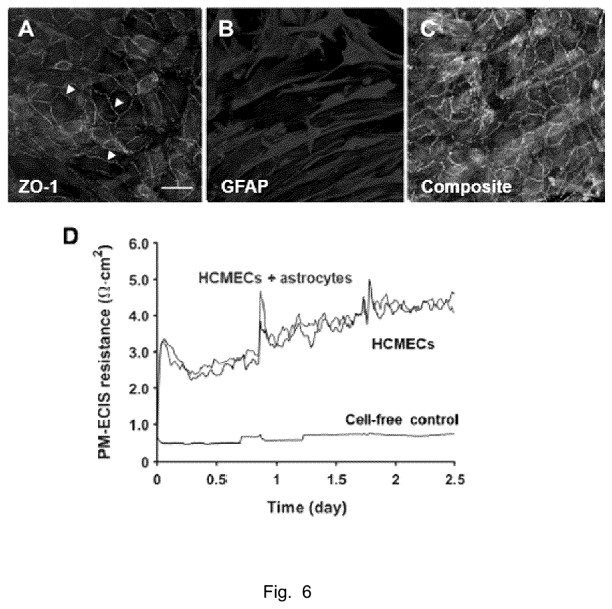

A significant advantage of PM-ECIS devices over traditional solid substrate ECIS devices is that porous membranes enable co-culture, as is typical for BBB modeling. To test the 6-well PM-ECIS inserts for this application, immortalized BBB endothelial cells and primary human astrocytes were cultured separately or together on the devices. Both cell types grew well when cultured separately on the devices, with HBMVECs forming confluent layers with junctional expression of ZO-1 (FIG. 6A) and astrocytes spreading on the membranes and expressing GFAP (FIG. 6B). Similarly, when co-cultured with the HBMVECs on the apical surface of the membrane (where the electrodes were patterned) and the astrocytes on the basal surface, both cell types grew as monolayers on the membranes and expressed their phenotypic markers (FIG. 6C).

ECIS resistance was measured for the monoculture and co-culture conditions in the 6-well, single electrode devices. As expected, brain endothelial cells (HCMEC/D3 line in FIG. 6D) grown on the apical surface of the membrane alone or in co-culture with astrocytes on the basal surface showed increasing 4 kHz PM-ECIS resistance over 2.5 days in culture, with typical minor fluctuations reflecting local cell movement on the single electrode (FIG. 6D). The resistance in co-cultures with astrocytes on the basal surface of the membrane was similar to that with monoculture, reflecting that the cells on the basal membrane surface do not contact the electrodes. The ability of membrane ECIS to isolate measurement of barrier function to only the barrier-forming cell monolayer is an important feature that is not possible with TEER.

Robust paracrine signaling between the apical and basal compartments of the PM-ECIS devices is expected, as the membrane porosity is retained over the bulk of the membrane. Direct cell contact via cell protrusions through the membrane pores would also be possible, except in regions with electrodes.

### PM-ECIS Devices Enable Measurement of Pericellular Resistances and Cell Membrane Capacitance

The total impedance between ECIS electrodes can be modeled as network of resistive and capacitive loads resulting from both the cell layer and non-cell impedances (i.e., capacitive interactions between electrodes and media) [see Giaever, I., Keese, C. R., 1991. Micromotion of mammalian cells measured electrically (cell motility/fibroblast behavior/nanometer motions/electrical measurements). Cell Biol. 88, 78-7900.]. Non-cell related parasitic impedances can be isolated and removed by measuring a baseline for each electrode pair before adding cells, as well as having a parallel cell-free control to measure electrode impedance drift over time. Pericellular resistance (Rb) across the tight junctions, a measure of true barrier integrity, can then be approximated via the impedance transfer function developed by Giaever and Keese by recording resistive and capacitive impedance components across a range of frequencies. This resistance is a linear measurement of the free space between the cells where small molecules can simply diffuse. The model also determines values for cell membrane capacitance (Cm) and basal pericellular impedance (a) between the cell layer and the electrodes; a is a function of the ratio between the average cell radius and the distance between the cell layer and the electrodes.

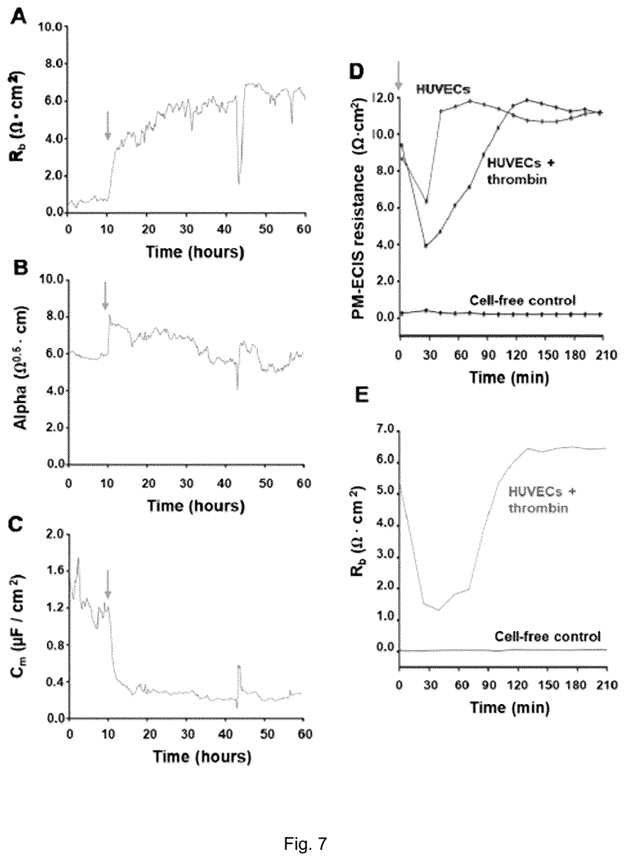

Rb, α, and Cm values were measured in HUVEC monolayers on the 6-well PM-ECIS inserts (FIG. 7). As expected, Rb rapidly increased with cell monolayer formation to a peak value of ˜6 Ω·cm² 60 hours post-seeding (FIG. 7A). Basal pericellular impedance, α, peaked at ˜8 Ω^{1/2}·cm during the first day of culture, and then gradually decreased to ˜6 Ω^{1/2}·cm after 60 hours (FIG. 7B), reflecting the reduction in the cells' sizes as they flattened and packed together to form a confluent monolayer. The values measured here for Rb and a were similar to those reported for solid substrate ECIS devices: 3.0-3.9 Ω·cm² and 5.3-5.4 Ω^{1/2}·cm for Rb and α, respectively. Initial values of Cm of ˜1.4 μF/cm² were observed, which is similar to that observed with commercial solid substrate ECIS systems (1.1-1.4 μF/cm²). Cm rapidly decreased over the first hours of culture coincident with the increases in Rb and a as the cells spread and became confluent, and then plateaued ˜0.3 μF/cm² (FIG. 7C). These trends also match those reported using solid substrate ECIS with epithelial cells and brain endothelial cells.

To confirm that the increase in measured resistance (and Rb) was due to cell barrier formation, cells were treated with thrombin to disrupt cell-cell junctions (cell culture media was replaced with fresh media containing 1 U/mL of thrombin from bovine plasma (Sigma) at the stimulus onset; fresh media without thrombin was used as a vehicle only control.) The addition of thrombin resulted in an acute resistance drop (FIG. 7D), indicating a temporary disruption in pericellular resistance, estimated by the Rb parameter (FIG. 7E); similar trends have been reported in commercial solid substrate ECIS systems. Control cultures in which only fresh media was added (vehicle control) also demonstrated a small drop in resistance (FIG. 7D); this is typical with media changes (e.g., FIG. 5A) and recovery was much quicker (˜15 min) than with thrombin treatment (˜120 min).

### PM-ECIS Devices Allow for Simultaneous Investigation of Permeability Coefficients and Cell Layer Resistance

An advantage of PM-ECIS is that the porous membrane allows for simultaneous measurement of permeability and electrical resistance. To demonstrate this and test their correlation, primary HBMVECs were seeded on PM-ECIS devices at densities of 5,000 cells/cm² or 30,000 cells/cm² and cultured for 4.5 days. Barrier resistances at 400 Hz, 4 kHz, and 40 kHz were monitored by PM-ECIS throughout the culture period, and FITC-dextran permeability assays were performed at days 1, 2, 3, and 5.

To measure barrier permeability, 1 mL of 50 μg/mL 10 kDa FITC-dextran in phosphate-buffered saline with calcium and magnesium (PBS+/+) was added to the apical side of a 6-well membrane PM-ECIS device, and 2 mL of PBS+/+ to the basolateral side. Volumes were chosen to match volume height and not induce pressure driven flow across the membrane. 100 μL samples were taken from the basolateral side (after pipette mixing) at 0, 5, 10, 20, and 30 minutes. 100 μL of PBS+/+ was replenished to each well after every sample. Permeability constants were calculated and modeled via the methods described by Hubatsch et al. [Hubatsch, I., Ragnarsson, E. G. E., Artursson, P., 2007. Determination of drug permeability and prediction of drug absorption in Caco-2 monolayers. Nat. Protoc. 2, 2111-9.]

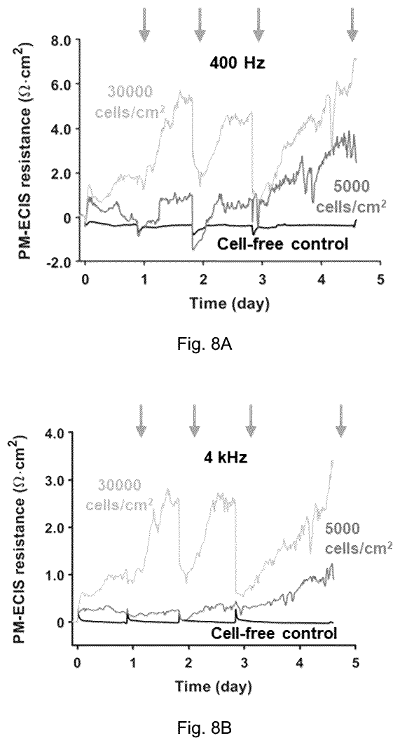

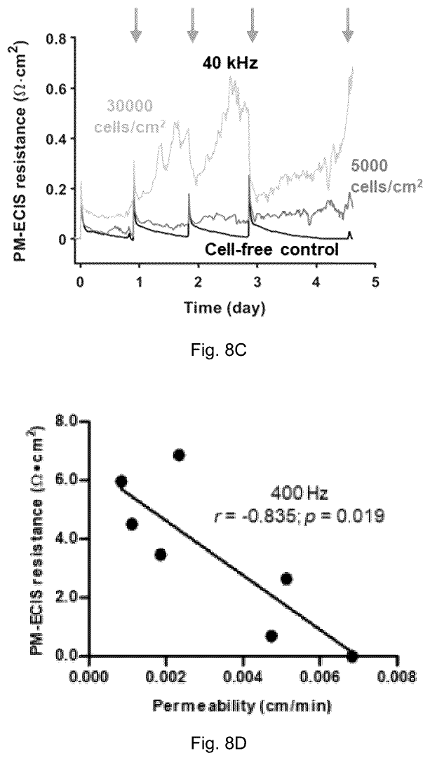

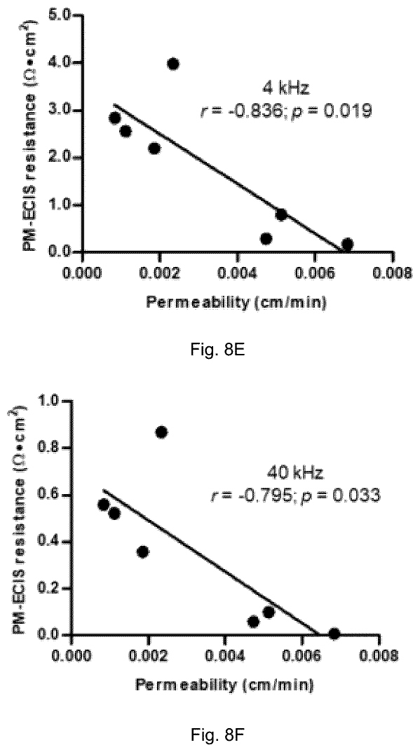

Differences in membrane coverage between the two seeding densities and with time in culture were reflected in higher PM-ECIS resistances for the higher density cultures at all frequencies and more rapid increases in resistances as the cells proliferated, spread, and formed barriers (FIG. 8A-C). The differences between the cultures initially seeded with 5,000 cells/cm² or 30,000 cells/cm² were also evident in the FITC-dextran permeability measurements: the day 4.5 permeability constants were 5.57×10⁻³±0.912×10⁻³ cm/min and 1.54×10⁻³±0.593×10⁻³ cm/min, respectively. Large drops in PM-ECIS resistances were observed after each permeability measurement, followed by lengthy lags recovery periods, which were sometimes incomplete (FIGS. 8A-C). This reflects the known disruptive effect of dextran on endothelial viability and function, and highlights the advantage of PM-ECIS for sensitive real-time measurement of barrier integrity without disruption of cultures. Nonetheless, significant negative correlations were observed between FITC-dextran permeability coefficients and PM-ECIS resistances at each of 400 Hz (FIG. 8D; r=−0.835, p=0.019), 4 kHz (FIG. 8E; r=−0.836, p=0.019), and 40 kHz (FIG. 8F; r=−0.795, p=0.033). Strong correlation between PM-ECIS and a tracer permeability assay is notable as it provides evidence that barrier function is similar between cells grown on the electrode (as measured by PM-ECIS) and directly on the bare membrane (as measured by a tracer assay), consistent with observations of uniform cell coverage (FIG. 4).

### Example 3: Validation of PM-ECIS and Comparison of PM-ECIS to TEER

PM-ECIS was validated across three different working electrode sizes (d=250, 500 and 750 μm). An electrode size-dependent response was demonstrated for monolayer growth and barrier formation for HUVECs and Caco-2 (epithelial) cells, and HUVEC barrier disruption with ethylene glycol-bis(β-aminoethyl ether)-N,N,N′,N′-tetraacetic acid (EGTA) and thrombin. This Example evidences a direct correlation between PM-ECIS and TEER measurements.

### Cell Culture

GFP-expressing HUVECs (AngioProteomie, cAP-0001GFP) were thawed and suspended in Endothelial Growth Medium (AngioProteomie, cAP-02). T75 flasks were pre-coated with 4 mL Quick Coating Solution (AngioProteomie, cAP-01) at room temperature for 5 min, then aspirated. HUVEC cell suspension (1×10⁶ cells) was transferred into the flask and placed overnight into a cell culture incubator (37° C., 5% CO₂) to allow cells to adhere. Medium was replaced with 15 mL fresh EGM the next day, and changed every other day thereafter, and cells grown to confluence. HUVECs were cultured for 6-7 days prior to detachment with 0.25% trypsin-EDTA (Life Technologies, 25200-056) for use in experiments. Inserts were coated with bovine plasma fibronectin (Sigma-Aldrich, F1141) at 50 μg/mL, 2 mL in each insert, and cells (P5) seeded at a density of 80 000 cells/cm² to achieve confluence by Day 1. In other experiments, Caco-2 cells (C2bbe1 clone) were cultured in Dulbecco's Modified Eagle Medium (DMEM) supplemented with 10% fetal bovine serum and 1% penicillin/streptomycin in T75 flasks. Inserts were coated with bovine plasma fibronectin (Sigma-Aldrich, F1141) at 50 μg/mL, 1 mL in each insert. Cells were seeded at a density of 100 000 cells/cm², and media changes performed daily.

### Device Fabrication

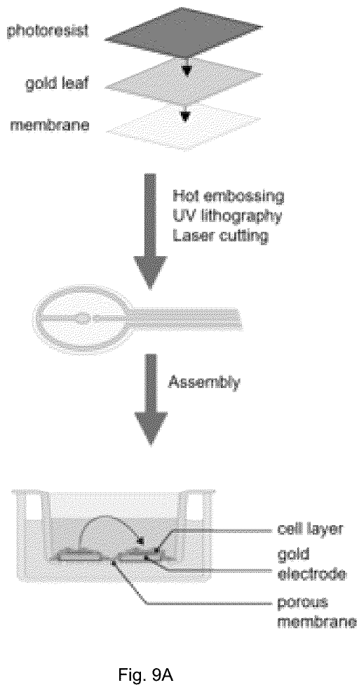

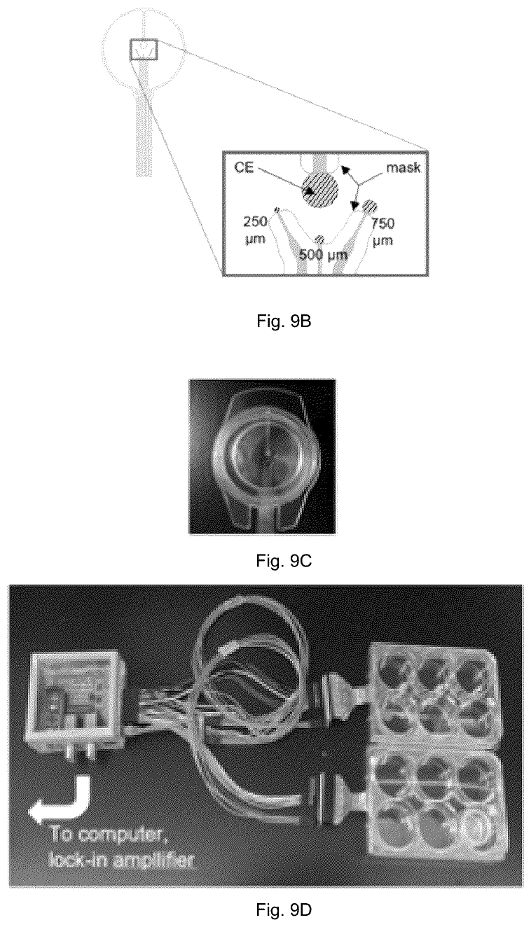

Each device comprised gold electrodes patterned onto a porous transparent PETE membrane filter (3.0 μm, 6×10⁵ pores/cm²; Sterlitech, 1300032) on which endothelial cells were cultured. Briefly, the device fabrication process involved thermal bonding of gold leaf (Gold Leaf Supplies, 2300/RUXX) to the membrane, followed by UV lithography to pattern the electrodes, and laser cutting of the electrode-patterned membrane to size and shape (FIG. 9A).

An electrode configuration was designed to test three different sizes of circular working electrode: 250 μm, 500 μm, and 750 μm diameters, with a circular counter electrode measuring 2 mm in diameter (FIG. 9B, 9C). Electrode diameters less than 250 μm were not tested, as only a few cells are sampled at that size and measurements are obscured by parasitic impedance from cables and measuring instruments at smaller diameters [J. A. Stolwijk, K. Matrougui, C. W. Renken, M. Trebak, Impedance analysis of GPCR-mediated changes in endothelial barrier function: overview and fundamental considerations for stable and reproducible measurements, Pflugers Arch. Eur. J. Physiol. 467 (2015) 2193-2218.]. The spacing of each working electrode with respect to the counter electrode was 1.5 mm, measured by taking the shortest edge-to-edge distance between each working electrode and the counter electrode. The exposed area of the patterned electrodes was defined by a tape mask. The total insert area was 3.83 cm².

### ECIS & TEER Measurements

The PM-ECIS devices were connected to a 24-channel multiplexer, which in turn connected to a phase-sensitive lock-in amplifier (LIA) and computer (FIG. 9D), The overall circuit for the ECIS system follows the set up described by Giaever and Keese. The LIA acts to both generate an alternating current signal, and receive signal measured across the cell layer. An alternating current signal of 1 V rms was generated at a specified frequency, connected to a 1 MΩ resistor to provide a 1 μA current. Cell-free devices were filled with culture medium and measurements taken over at least 12 hours to establish baseline impedance. The voltage drop across the device containing cells grown on electrodes was detected and measured by the LIA, which allows for small AC signals to be resolved in high noise systems. A cell-free baseline was established beforehand using cell-free devices filled with culture medium and impedance measurements taken over the course of at least 12 hours prior to cell seeding. LIA outputs including voltage drop and phase angle were used to calculate and plot impedance and resistance values.

TEER was measured using a Millicell ERS volt-ohm meter (World Precision Instruments, New Haven, CT) according to the manufacturer's instructions.

### Immunostaining

Cells were fixed in methanol for 10 min at −20° C., washed three times with PBS, and blocked with 3% BSA (Sigma-Aldrich, 10735086001) for 20 min at 37° C. Cells were stained with primary antibodies (1:100) diluted in 3% bovine serum albumin in PBS for 1 hour at 37° C. Following incubation, cells were washed three times with PBS and blocked using 10% goat serum (Sigma-Aldrich, G9023) in PBS for 1 hour at room temperature. Secondary antibodies (1:200) diluted in 10% goat serum were applied for 1 hour at room temperature. After washing three times with PBS, nuclei were stained with 1:1000 Hoechst 33342 (Sigma-Aldrich, B2261) for 5 min at room temperature. Primary antibodies were rabbit polyclonal VE-cadherin (Abcam, ab33168) and mouse monoclonal ZO-1 (Thermo Fisher Scientific, 33-9100); secondary antibodies were goat anti-mouse IgG (H+L) Alexa Fluor 647 (Life Technologies, A-21236) and goat-anti rabbit IgG (H+L) Alexa Fluor 568 (Thermo Fisher Scientific, A-11011). Image acquisition was performed using an Olympus FV3000 confocal laser scanning microscope or Olympus BX51 inverted fluorescence microscope as indicated.

### EGTA and Thrombin Treatment Assays

Cells were incubated in 5 mM EGTA (Sigma-Aldrich, E3889) diluted in Endothelial Growth Medium at 37° C. and 5% CO2 for 2 hours, with ECIS measurements taken every 15 min. After 2 hours, EGTA was removed and replaced with fresh growth medium. For transient disruption of the endothelial barrier, cells were stimulated with 1 U/mL of thrombin from bovine plasma (Sigma-Aldrich, T6634) diluted in Endothelial Growth Medium at 37° C. and 5% CO2 for 2 hours, with measurements taken every 5 min.

### NaCl Concentration Curve

Cell-free PM-ECIS 6-well inserts were filled with varying concentrations of NaCl (0.005, 0.01, 0.1, 1 M) and measurements taken for each concentration by chopstick TEER and ECIS. Values reported for TEER are resistance as measured by the Millicell ERS volt-ohm meter without adjustment. ECIS values are resistance values measured without adjustment.

Comparison TEER Vs. ECIS Calculations

TEER values were calculated using TEER=(Rcell−Rcell-free)×surface area of insert. ECIS values were calculated using ΔR=Rcell−Rcell-free. TEER and ECIS values were normalized to the 6 h time point for each of the experimental runs, with up to three devices tested per run.

### Characterization of Cells Grown on ECIS Membrane Device

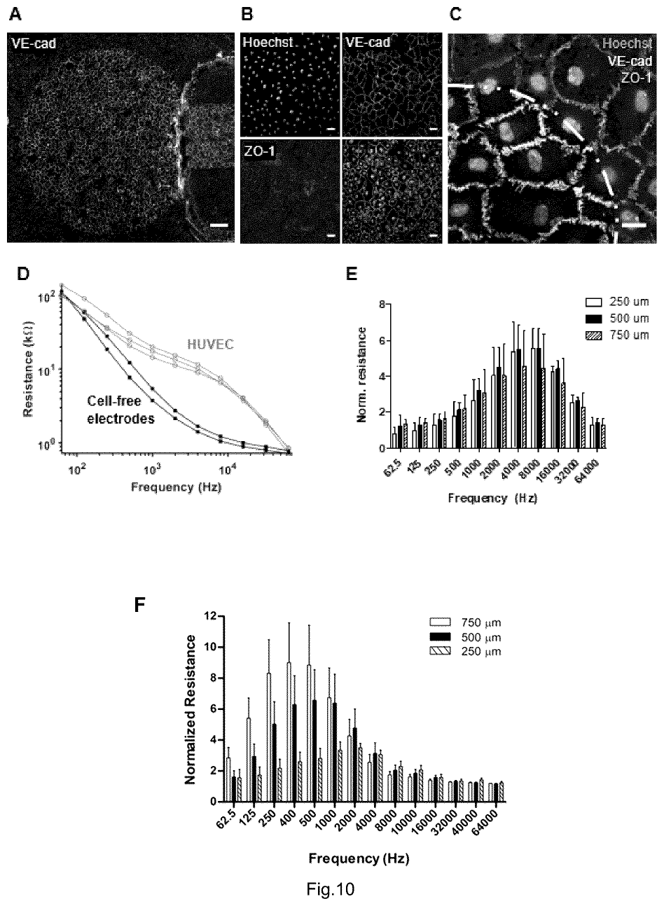

To assess the ability of endothelial cells to form confluent monolayer on different sizes of PM-ECIS working electrodes, GFP-expressing HUVECs were seeded at 80 000 cells/cm² and cultured for five days in devices with one counter electrode and three different working electrode sizes of 250, 500, and 750 μm diameter. Each working electrode measured its corresponding cell population independently from the other working electrodes in the same device. Immunostaining confirmed that the PM-ECIS devices supported the growth of confluent HUVEC monolayers, with VE-cadherin continuously distributed around the cell peripheries (FIG. 10A) and punctate ZO-1 membrane expression across the cell layer (FIG. 10B), consistent with previous observations of HUVEC monolayers on tissue culture-treated polystyrene and porous membrane substrates. Notably, the HUVEC monolayer spanned the electrode-membrane boundary with no visible effect on morphology or expression of junctional proteins compared to surrounding cells (FIG. 10C).

To validate PM-ECIS vs. conventional ECIS measurements in the literature for endothelial cells, frequency scans were performed on HUVECs four days post-confluence, with measurements taken at f=62.5, 125, 250, 500, 1 000, 2 000, 4 000, 8 000, 16 000, 32 000, and 64 000 Hz. PM-ECIS yielded the expected frequency-resistance relationship for cell-covered vs. cell-free electrodes, with the cell layer contributing to a characteristic increase in signal over the mid-range frequencies compared to cell-free electrodes [Stolwijk, J. A et al. Impedance Analysis of GPCR-Mediated Changes in Endothelial Barrier Function: Overview and Fundamental Considerations for Stable and Reproducible Measurements. Pflugers Arch. Eur. J. Physiol. 2015, 467, 2193-2218; Arndt, S. et al. Bioelectrical Impedance Assay to Monitor Changes in Cell Shape during Apoptosis. Biosens. Bioelectron. 2004, 19, 583-594] (FIG. 10D). Normalized resistance values of HUVECs on PM-ECIS devices peaked at ˜4 kHz for all electrode sizes, consistent with the 4 kHz measurement frequency conventionally used for HUVEC monolayers in solid substrate ECIS setups (FIG. 10E).

To validate PM-ECIS vs. conventional ECIS measurements in the literature for epithelial cells, Caco-2 cells were seeded at 100 000 cells/cm² and cultured for five days in devices with one counter electrode and three different working electrode sizes of 250, 500, and 750 μm diameter. Epithelial barrier formation was monitored by PM-ECIS electrodes over 4 days (measurement frequencies=400, 1000, 4000, 10 000, 40 000 Hz). Frequency scans (62.5, 125, 250, 400, 500, 1000, 2000, 8000, 10 000, 16 000, 32 000, 40 000, 64 000 Hz) were performed on Day 2 after cell seeding. Analysis of frequency scans and normalized resistance values indicated that resistance measurements at 1000 Hz allow for sensitive detection of the Caco-2 barrier on 250 μm electrodes, whereas 400 Hz is a more suitable frequency for 750 μm and 500 μm electrodes (FIG. 10F).

These findings are consistent with previous literature with solid-substrate ECIS where low to mid-range frequencies (400 Hz-1000 Hz) have been used for measuring Caco-2 and other epithelial barriers, as well as a previous study that found that decreasing electrode diameter corresponds with peak normalized resistance shifting to higher frequencies [Charrier, L. et al. ADAM-15 Inhibits Wound Healing in Human Intestinal Epithelial Cell Monolayers. Am. J. Physiol.—Gastrointest. Liver Physiol. 2005, 288, G346-G353; Laroui, H et al. Dextran Sodium Sulfate (Dss) Induces Colitis in Mice by Forming Nano-Lipocomplexes with Medium-Chain-Length Fatty Acids in the Colon. PLoS One 2012, 7 (3); Merlin-Zhang, O. et al. In Vitro Intestinal Epithelial Wound-Healing Assays Using Electric Cell-Substrate Impedance Sensing Instrument. Bio-protocol 2019, 9 (17); Yamaguchi, E. et al. Electric Cell-Substrate Impedance Sensing (ECIS) as a Platform for Evaluating Barrier-Function Susceptibility and Damage from Pulmonary Atelectrauma. Biosensors 2022, 12, 390; Lai, Y.-T. et al. Effects of Electrode Diameter on the Detection Sensitivity and Frequency Characteristics of Electric Cell-Substrate Impedance Sensing. Sensors Actuators B Chem. 2019, 288, 707-715.]

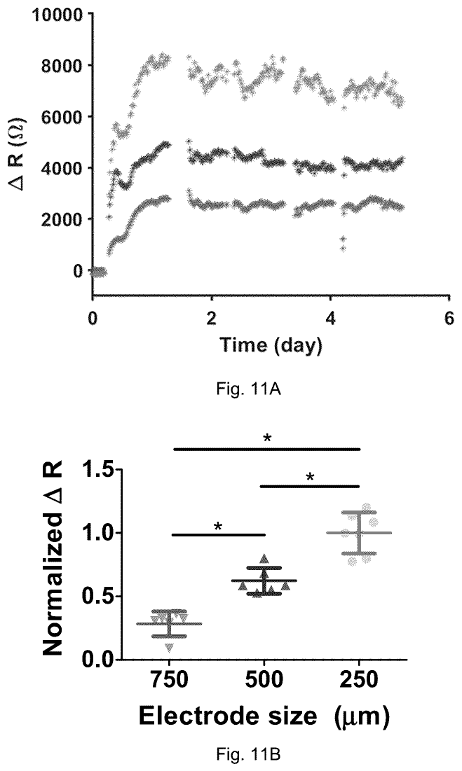

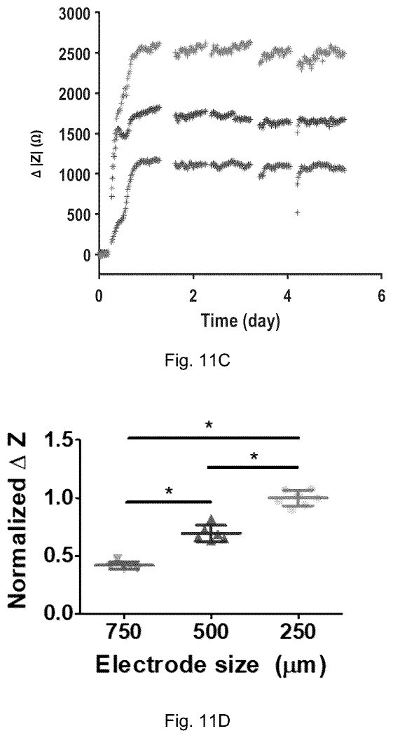

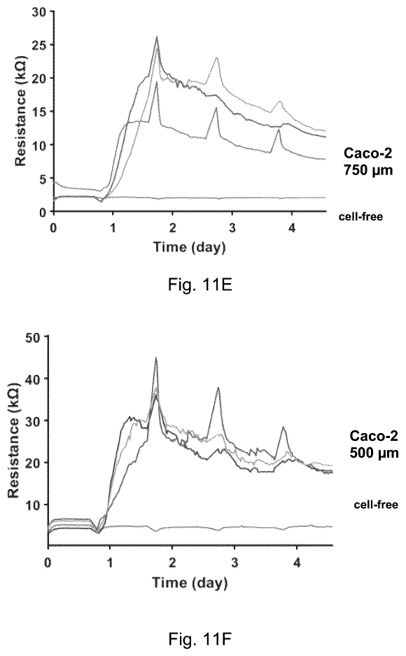

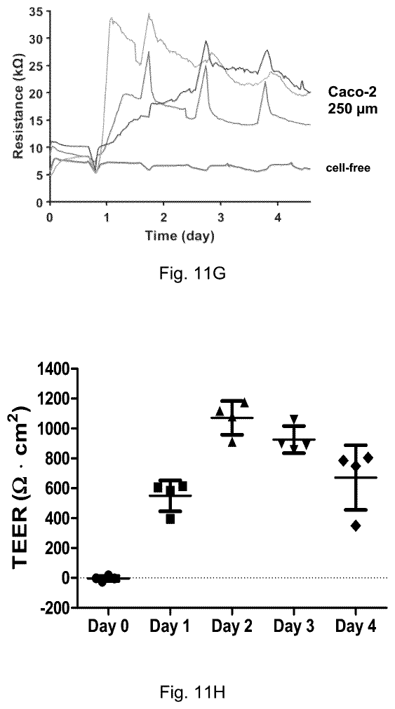

PM-ECIS measurements indicated confluent Caco-2 monolayer formation and peak resistance by Day 2 (FIG. 11E, 11F, 11G), which was further confirmed by chopstick TEER measurements in the same devices (FIG. 11H).

### Effect of Electrode Size on PM-ECIS Measurements of Monolayer Growth and Barrier Integrity

The contribution to overall impedance by the electrode-solution interface is proportional to 1/r², where r is radius of the working electrode; thus, impedance is inversely proportional to electrode size, with smaller working electrodes expected to be more sensitive than larger ones [J. A. Stolwijk, K. Matrougui, C. W. Renken, M. Trebak, Impedance analysis of GPCR-mediated changes in endothelial barrier function: overview and fundamental considerations for stable and reproducible measurements, Pflugers Arch. Eur. J. Physiol. 467 (2015) 2193-2218.]. To test this in PM-ECIS devices, growth of HUVECs was monitored on three electrode sizes (d=250, 500, 750 μm) over 5 days. To assess electrode size effects on PM-ECIS measurement of barrier formation, change in normalized resistance at 4 kHz was measured throughout the culture period and compared between electrode sizes. Resistance increased over the first ˜2 days of culture on all electrode sizes, with the change in resistance being inversely proportional to the electrode size (FIG. 11A). From Day 3 to 5 of culture, resistance values plateaued for all electrode sizes, indicating formed barriers (FIG. 11A). Endpoint analysis on Day 5 confirmed a significant effect of electrode size on normalized resistance (p<0.001), with the greatest change on the smallest (250 μm) electrodes (FIG. 11B). To evaluate whether the same trends applied to cell spreading and proliferation across the PM-ECIS electrodes, impedance was measured at 40 kHz (FIG. 11C), and there was a significant effect of electrode size on normalized impedance (p<0.001), with the greatest change on the smallest electrodes (FIG. 11D).

The effect of electrode size on PM-ECIS measurements of Caco-2 epithelial cell monolayer growth and barrier integrity was also tested. Resistance measurements at 400 Hz for 750 μm (FIG. 11E) and 500 μm (FIG. 11F) electrodes showed characteristic increase over the first ˜2 days of culture, with change in resistance being inversely proportional to the electrode size. Resistance measurements at 1000 Hz for 250 μm electrodes showed similar increase over the first ˜2 days of culture (FIG. 11G). On all electrode sizes, resistance values decreased from Day 3 to 5 of culture, indicating reduced barrier integrity with time in culture (FIG. 11E-G); this temporal trend in barrier resistance was validated by TEER measurements of the same cultures (FIG. 11H)

These trends on PM-ECIS devices are consistent with those reported on traditional ECIS.

### Measurement of Response to EGTA and Thrombin Barrier Disruption

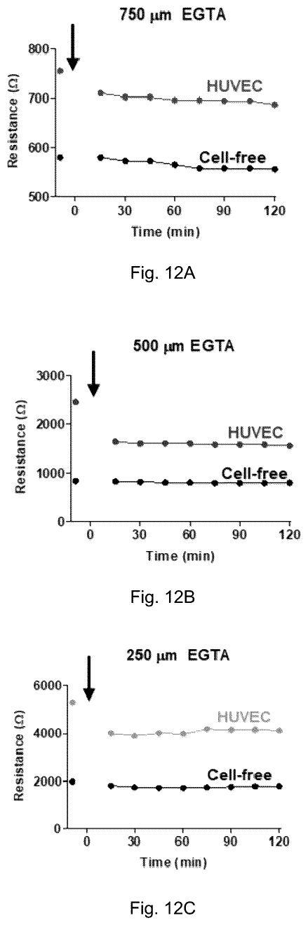

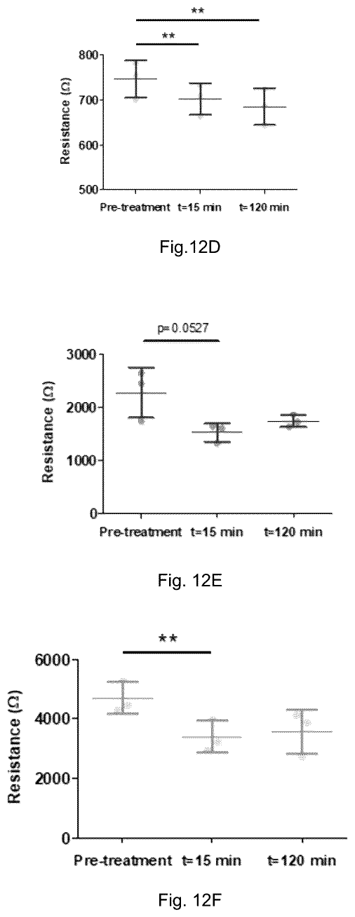

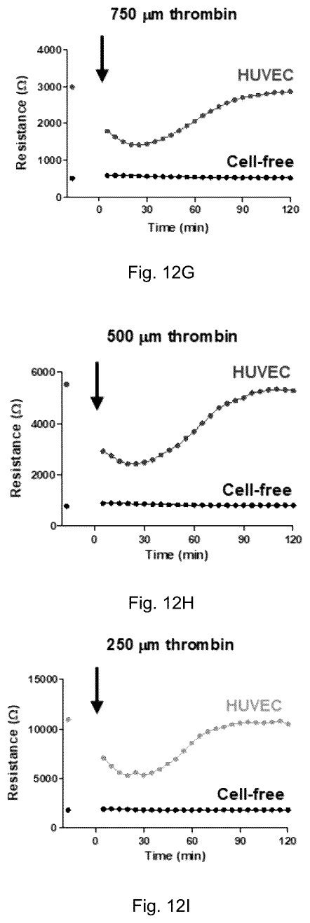

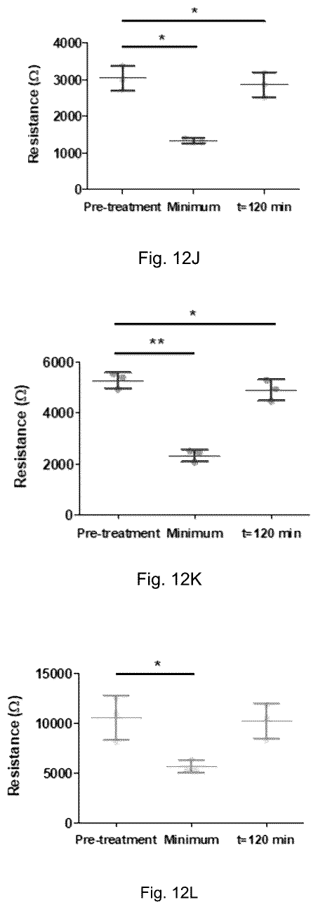

To assess the effect of electrode size in PM-ECIS response to barrier disruption, a confluent monolayer of HUVECs was subjected to 2-hour treatment with EGTA. EGTA disrupts cell barriers by chelating Ca2+ ions to destabilize tight junctions. Therefore, a decrease in barrier integrity, and by extension, ECIS resistance, would be expected after EGTA treatment and would expect to persist for >120 minutes until junctions can re-form [Panou, D. A. et al. Epithelium Dynamics Differ in Time and Space When Exposed to the Permeation Enhancers Penetramax and EGTA. A Head-to-Head Mechanistic Comparison. Front. Drug Deliv. 2023, 3 (August), 1-16; Moztarzadeh, S. et al. Cortactin Is in a Complex with VE-Cadherin and Is Required for Endothelial Adherens Junction Stability through Rap1/Rac1 Activation. Sci. Rep. 2024, 14 (1), 1-18.] Barrier disruption with EGTA treatment resulted in a drop in resistance within 15 minutes, measured at 4 kHz for all electrode sizes (FIG. 12A-F; p<0.01 for 250 and 750 μm electrodes and p=0.0575 for 500 μm electrodes). While all electrodes sizes were sensitive to barrier disruption, the reduction in barrier resistance was significantly greater with the 250 μm (p<0.001) and 500 μm (p<0.05) electrodes than the 750 μm electrodes (FIG. 12D-F).

The effect of electrode size in PM-ECIS response to barrier disruption was also tested by treatment of a confluent monolayer of HUVECs with thrombin. Thrombin disrupts cell barriers by causing transient cell contraction. Therefore, a transient decrease in barrier integrity, and by extension, ECIS resistance, would be expected after thrombin treatment, with recovery of the barrier (and by extension, return to pre-treatment ECIS resistance values) within 120 minutes [Arce, F. T. et al. Regulation of the Micromechanical Properties of Pulmonary Endothelium by S1P and Thrombin: Role of Cortactin. Biophys. J. 2008, 95 (2), 886-894; Aslam, M. et al. CAMP Controls the Restoration of Endothelial Barrier Function after Thrombin-Induced Hyperpermeability via Rac1 Activation. Physiol. Rep. 2014, 2 (10), 1-13.]. Barrier disruption with thrombin treatment resulted in an immediate drop in resistance, measured at 4 kHz for all electrode sizes (FIG. 12G-L; p<0.01 pre-treatment vs. minimum resistance for all electrode sizes). While all electrodes sizes were sensitive to barrier disruption, the reduction in barrier resistance was inversely proportional to electrode size (FIG. 12J-L). Within 120 minutes of thrombin treatment, resistances on all electrode sizes returned to or close to pre-treatment values, indicated sensitive measurement of restoration of the barrier after transient disruption (FIG. 12G-L).

### Comparison of PM-ECIS to TEER

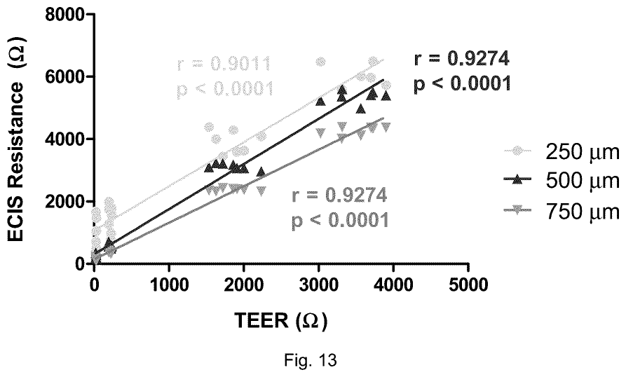

To further validate PM-ECIS, comparison to chopstick TEER was initially performed using NaCl concentration curve measurements, a method previously used by Cacopardo et al. for impedimetric system assessment [L. Cacopardo, et al. Real-time cellular impedance monitoring and imaging of biological barriers in a dual-flow membrane bioreactor, Biosens. Bioelectron. 140 (2019) 1-17.]. Cell-free PM-ECIS 6-well inserts were filled with varying concentrations of NaCl (0.005, 0.01, 0.1, 1 M) and resistance measured by chopstick TEER and ECIS. As expected, the resistance measured by both TEER and PM-ECIS was NaCl concentration-dependent, with statistically significant correlation between the two methods for all three electrode sizes (FIG. 13; r>0.9; p<0.0001).

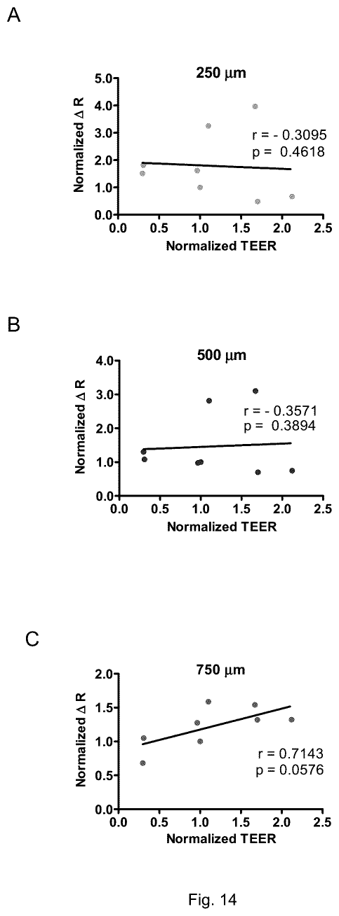

Having shown strong correlation between ECIS and TEER for NaCl solutions, correlation between the two methods for endothelial cell monolayers was next assessed. HUVECs were seeded in devices with all three electrode sizes, and normalized TEER and ECIS measurements were taken over 48 h post-seeding. ECIS measurements from the 250 and 500 μm diameter electrodes were not correlated to TEER values obtained for same cell monolayer (FIG. 14A, 14B; p=0.46 and p=0.39, respectively). This likely reflects differences in the spatial scales to which the two methods are sensitive: TEER measures resistance across the entire cell monolayer and media compartments, whereas small PM-ECIS electrodes sample a smaller number of cells with high sensitivity to local changes in the cell monolayer (FIGS. 11, 12). Consistent with this, normalized ECIS resistance for HUVECs grown on the largest 750 μm electrodes was positively and significantly correlated to normalized chopstick TEER values (FIG. 14C; p=0.058).

The trade-off between signal sensitivity and cell population sampled with small single working electrodes can be addressed with multiple small electrodes measuring in parallel to a common counter electrode. Such a design can increase the sampling area albeit with a small reduction in sensitivity compared to a single working electrode for confluent monolayers normalized to cell-free electrodes. Single electrodes do, however, provide a higher magnitude signal in response to, and better resolution of slope differences over, the various stages of cell adhesion and movement when measuring rate of change in impedance over time [Hung Y. H. et al. ECIS Based Electric Fence Method for Measurement of Human Keratinocyte Migration on Different Substrates, Biosensors. 12 (2022).]. This suggests a particular relevance of the single electrode configuration tested in this study to assays where time resolution of cell response to a treatment condition is being evaluated.

### Example 4: Use with Hydrogel and in a Blood-Brain Barrier Model

Effect of Collagen Hydrogel on ECIS Resistance Vs. Chopstick TEER

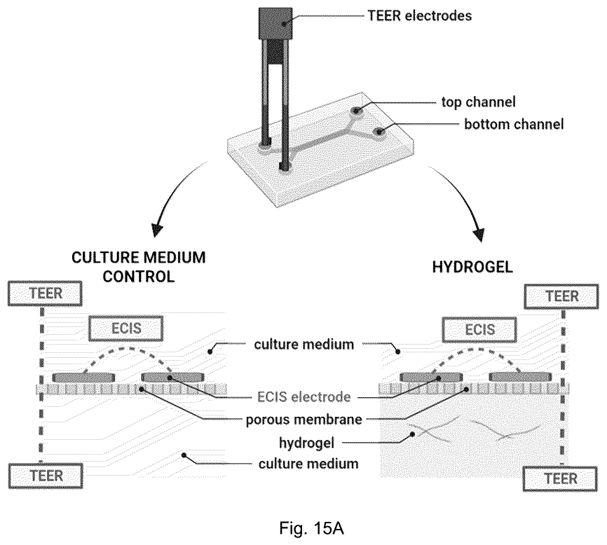

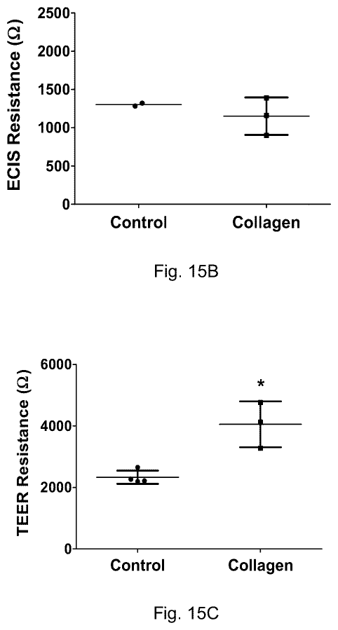

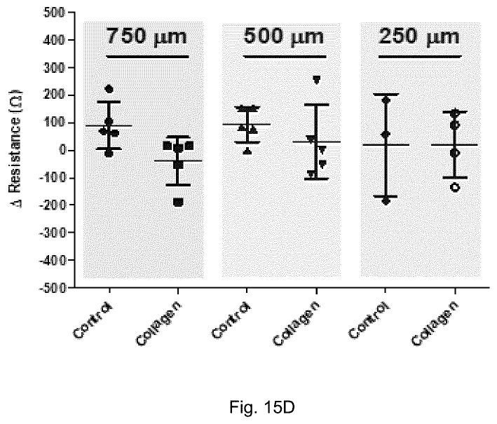

Organ-on-a-chip configurations of membrane-based microfluidic systems often include a hydrogel in the abluminal channel, the presence of which alone could affect cell barrier impedance measurements. To test this, the resistances of cell-free membranes were measured by ECIS and chopstick TEER in devices containing DMEM in the top channel and either DMEM (control) or collagen hydrogel (collagen) in the bottom channel (FIG. 15A). Importantly, presence of hydrogel alone in the bottom channel did not influence ECIS resistance (FIG. 15B), compared to TEER measurements in same devices (FIG. 15C), which showed a difference of 1723) (p=0.006) between control and hydrogel conditions. The resistance measurements performed by the ECIS electrodes were robust to the presence or absence of hydrogel in the bottom channel across all electrode sizes, with no significant difference between the control and hydrogel conditions (FIG. 15D).

### Blood-Brain Barrier Model in Microfluidic ECIS Platform

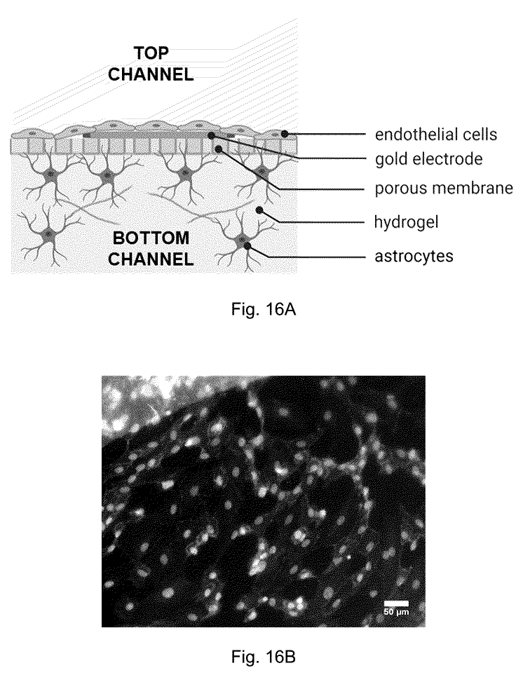

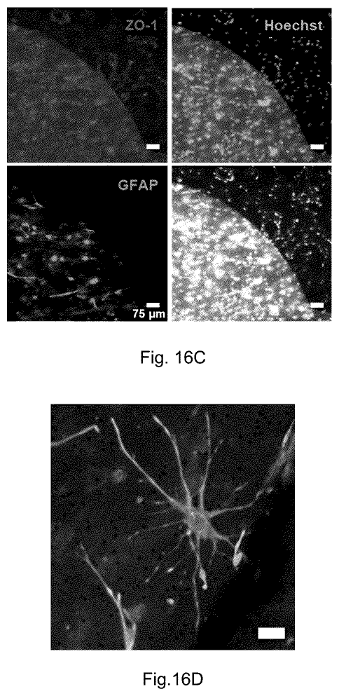

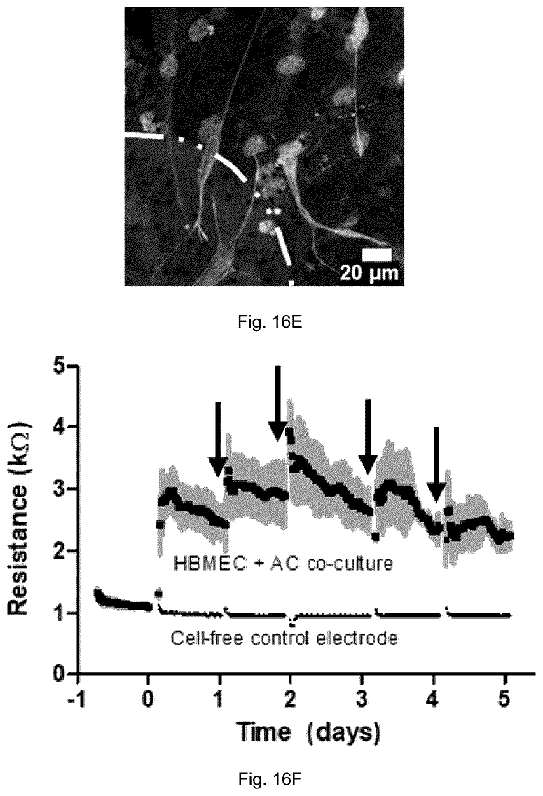

Having established that ECIS resistance measurements in the microfluidic platform were unaffected by the presence of a hydrogel, the platform was tested in a relevant 3D co-culture model of the blood-brain barrier. HBMECs were grown on the luminal side of the porous membranes with the ECIS electrodes, and primary human astrocytes embedded in collagen hydrogel were grown in the abluminal channel (FIG. 16A) for up to 5 days. HMBECs were evident on the porous membrane and electrodes on the luminal side by their expression of the tight junction zonula occludens-1 (ZO-1) (FIG. 16 B,C), while astrocytes expressing glial fibrillary acidic protein (GFAP) were evident on the abluminal membrane surface and spanned the membrane-electrode border (FIG. 16 D,E). The platform supported BBB co-culture over the course of 5 days, reaching peak barrier integrity on Day 3 post-seeding as measured by ECIS resistance at 4 kHz using the 500 μm diameter electrodes (FIG. 16F).

### Example 5: Microfluidic ECIS Measurements Upon Application of Fluid Flow

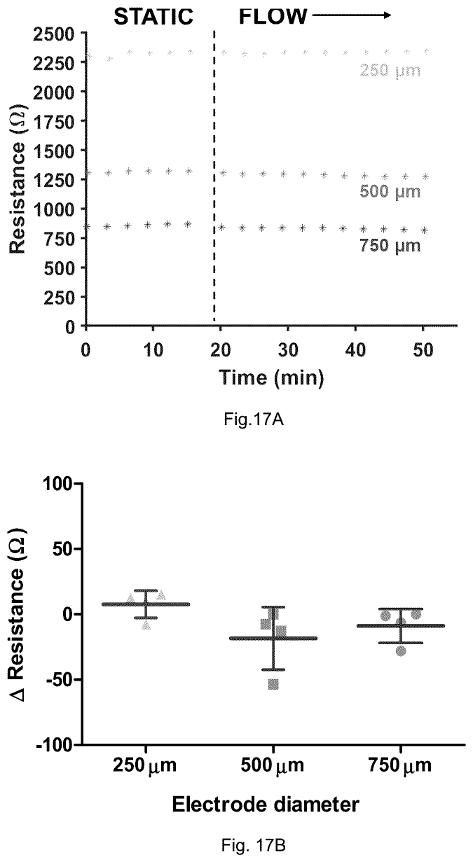

To first validate that ECIS measurements were not affected by fluid flowing over the electrodes on the porous membrane, PBS was introduced into the luminal channel of blank microfluidic ECIS devices using a syringe pump at a flow rate of 1.3 mL/min (equivalent to a wall shear stress of 5 dyn/cm²). ECIS measurements were taken every 3 min before and after introduction of fluid flow for up to 50 minutes. Measurements in the blank devices with PBS indicated that ECIS resistance measurements were not affected by the application of fluid flow (FIG. 17A), with no significant differences between the pre-flow and post-flow values for any of the electrode sizes (FIG. 17B).

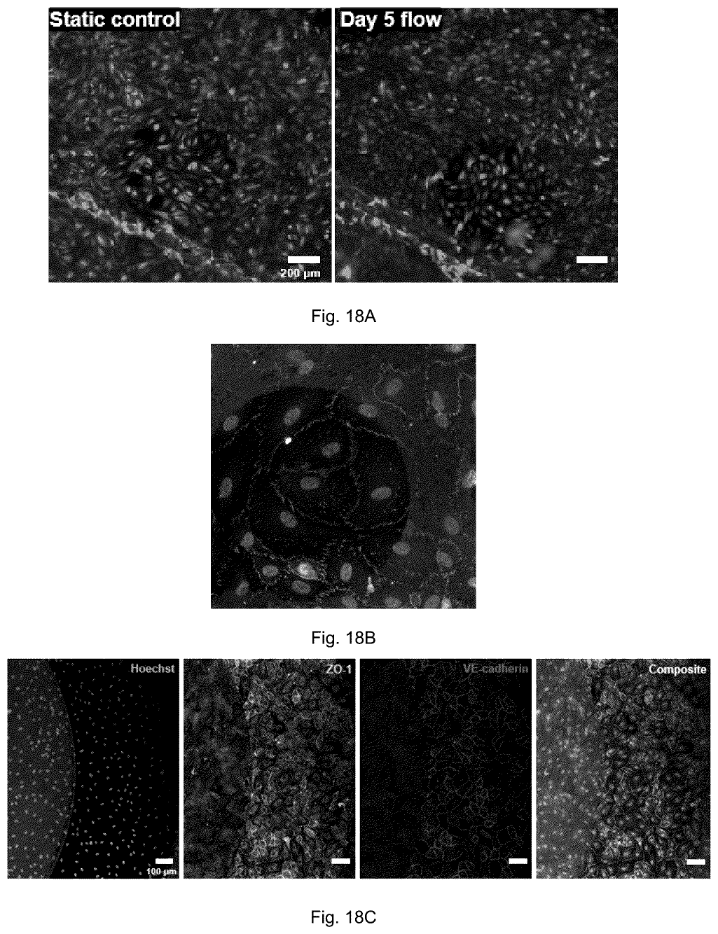

To test the platform's capability for real-time ECIS measurement on perfused cultured cells, GFP-expressing HUVECs were seeded into the luminal channels, allowed to reach confluence over 2 days under static conditions, and then subjected to a perfusion flow rate of 20 μL/min (0.01 dyn/cm² wall shear stress). Parallel control cultures were maintained under static conditions. Live fluorescence imaging confirmed that cells perfused for up to 5 days remained viable and adhered to the porous membrane and electrodes comparably to cells grown under static conditions for the same duration (FIG. 18A). Immunostaining for ZO-1 and VE-cadherin demonstrated that the perfused endothelium formed a confluent monolayer on the working (FIG. 18B) and counter (FIG. 18C) electrodes.

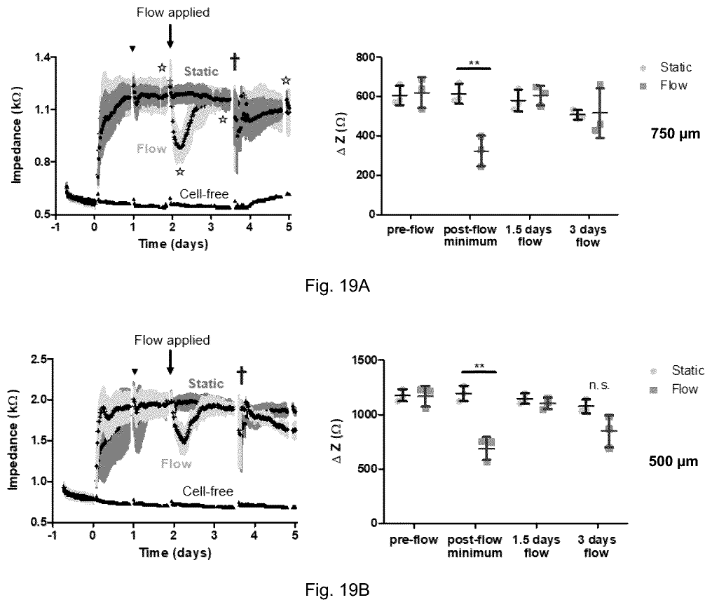

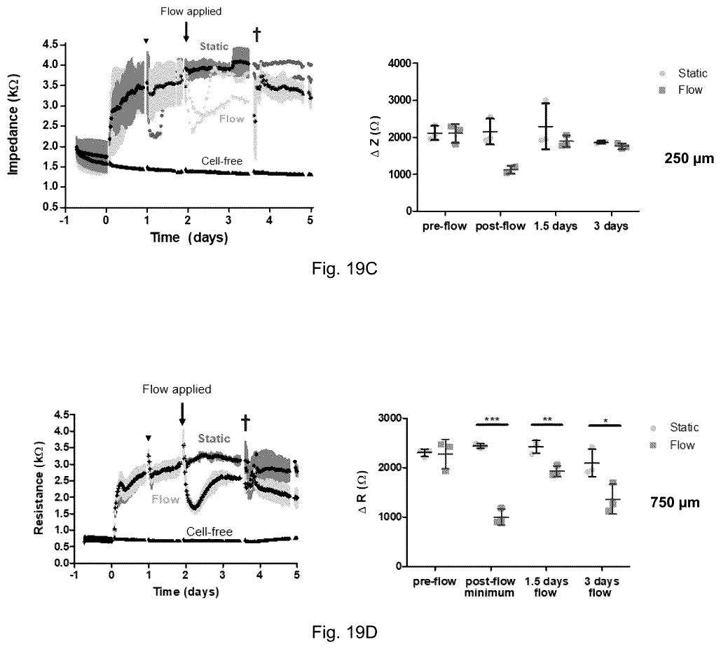

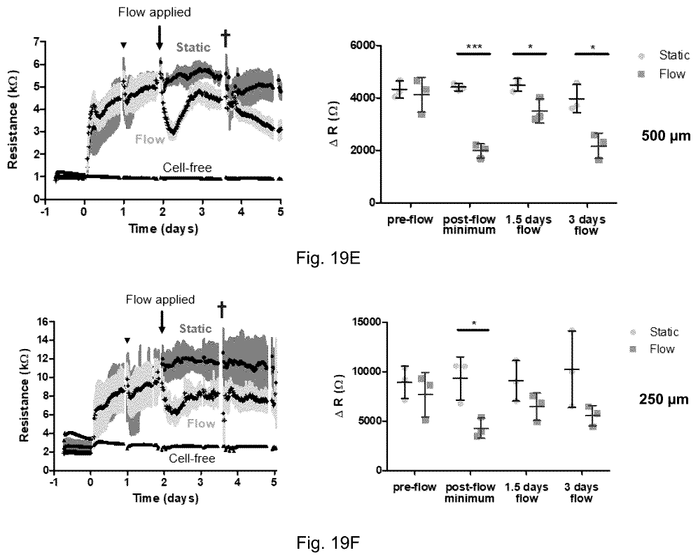

ECIS measurements were made continuously using three electrode sizes in each device prior to cell seeding, for 2 days post-seeding under static culture, and then for 3 days of either perfusion or static culture. In all cases, ECIS impedance (40 kHz) rapidly increased after cell seeding as the cells adhered and spread to form confluent monolayers, typically within one day of seeding (FIG. 19A-C). ECIS resistance (4 kHz) also rose rapidly in this period but did not fully plateau after two days of static culture, suggesting cell junctions were continuing to mature at this time point (FIG. 19D-F).

The initiation of perfusion at Day 2 resulted in immediate significant decreases in impedance (FIG. 19A-C) and resistance (FIG. 19D-F) relative to static control cultures (p<0.01 for 750 and 500 μm electrode impedance at 40 kHz; p<0.05 for all electrode sizes for resistance at 4 kHz), indicating rapid flow-induced cell monolayer remodeling that decreased cell coverage and barrier integrity. Within 24 hours of flow initiation, ECIS impedances in perfused cultures returned to levels that were not significantly different from those of static control cultures (FIG. 19A-C; p>0.3 for all electrode sizes on Day 1.5 after flow), indicating recovery of cell coverage on the ECIS electrodes and membranes. Impedances remained relatively constant for the duration of the culture period, even when flow was paused briefly as the syringes were refilled with media at Day 3.5 (FIG. 19A-C). Recovery and maintenance of cell coverage detected by ECIS impedance measurement was consistent with the endpoint immunostaining images showing confluent cultures (FIG. 18A-C).

In contrast to impedance measurements, ECIS resistances measured at 4 kHz only partially recovered after flow initiation and trended lower over the 3 days of perfusion suggesting continued remodeling of the cell monolayer junctions over this period. In particular, resistance values measured by the larger 500 μm and 750 μm electrodes were significantly lower in the perfused cultures than in static both 1.5 days and 3 days after flow initiation (FIG. 19D, E; p<0.05). Similarly, resistances of perfused cultures measured by the smaller 250 μm electrodes trended lower than those of static cultures, but the differences were not significant (FIG. 19F), in part because focal measurements with smaller electrodes result in great variability compared with more global measurements with larger electrodes.

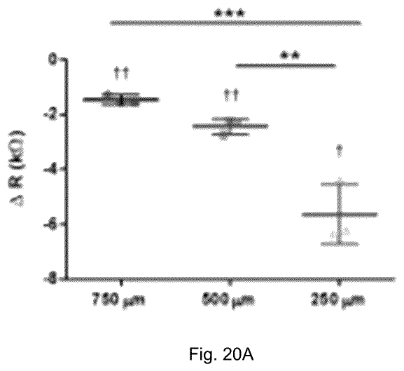

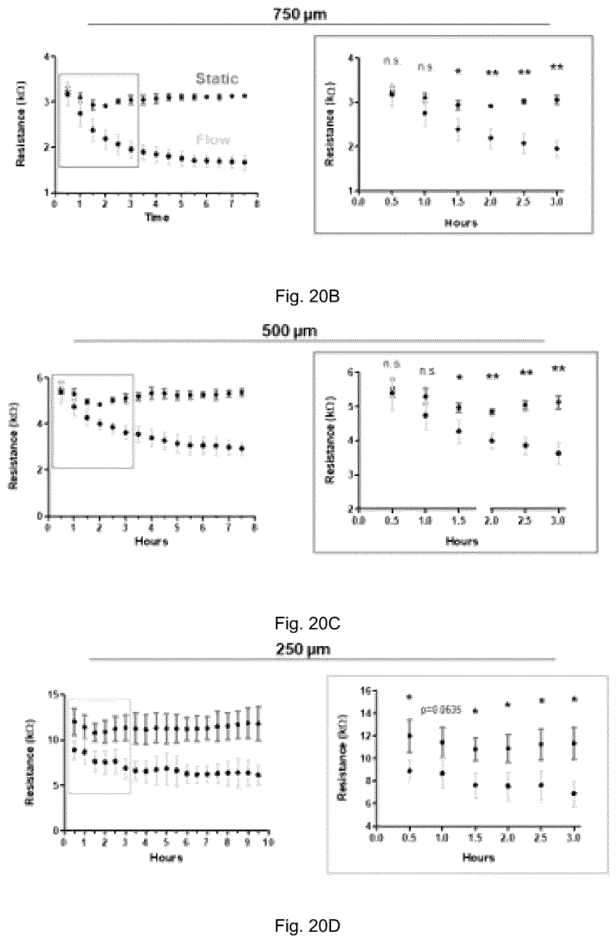

Of note, the 250 μm electrodes were the most sensitive to changes in barrier resistance, both temporally and with respect to magnitude change in resistance. All three of the electrode sizes detected a significant decrease in resistance compared to static control at the post-flow minimum (FIG. 20A), with 250 μm electrodes showing the greatest magnitude decrease compared to 500 μm (p<0.01) and 750 μm (p<0.001). The 250 μm electrodes were also most sensitive to immediate change in barrier integrity following flow, with a significant decrease in resistance at 4 kHz (p=0.039) compared to static control detected within 30 minutes of applying fluid flow (FIG. 20B); for 500 and 750 μm electrodes decreased resistance was not detected until 1.5 hours post-flow (p=0.034 and p=0.026, respectively) (FIG. 20C,D).

As can be seen therefore, the examples described above and illustrated are intended to be exemplary only. The scope is indicated by the appended claims.

What is claimed is:

1. A method of manufacturing an electrical cell-substrate impedance sensing (ECIS) membrane electrode comprising:
heat-bonding a biocompatible electrically conductive sheet directly to a porous membrane to form a laminate having an electrically conductive surface and a porous membrane surface; and
forming electrode components by etching the electrically conductive surface using photolithography techniques.

2. The method of claim 1, wherein forming the electrode components using photolithography techniques comprises:
a) hot embossing a gold- or gold alloy-coated membrane to a rigid or substantially rigid substrate;
b) applying photoresist film over the gold- or gold alloy-coated membrane;
c) placing a photomask on top of the photoresist, and exposing the photoresist to UV light;
d) removing unexposed photoresist and washing to remove any residue; and
e) using gold etching solution to remove gold/gold alloy not covered by the exposed photoresist, and washing to remove etching solution and photoresist.

3. The method of claim 1, wherein forming the electrode components using photolithography techniques comprises:
a) hot embossing a gold- or gold alloy-coated membrane to a rigid or substantially rigid substrate;
b) applying a dry film photoresist by lamination at 20-25° C.;
c) placing a photomask on top of the photoresist, and exposing the photoresist to UV light;
d) removing unexposed photoresist using a sodium carbonate in water solution and washing with isopropanol to remove any residue;
e) removing gold/gold alloy not covered by the exposed photoresist using gold etching solution and washing with acetone to remove etching solution and photoresist; and
optionally, f) removing the remainder of the photoresist using NaOH.

4. The method of claim 3, wherein:
the photoresist is exposed to UV light for 8-10 seconds;
the sodium carbonate in water solution is a 1% sodium carbonate in water solution; and/or
the NaOH comprises 0.5 NaOH.

5. A method of manufacturing an electrical cell-substrate impedance sensing (ECIS) membrane electrode comprising:
(a′) Laminating a thermoplastic film to a substrate;
(b′) Laminating porous membrane coated in gold or a biocompatible gold alloy to the thermoplastic film layer of the laminate of step (a′), optionally wherein (a′) and (b′) are performed concurrently;
(c′) Hot embossing the laminate of step (b′) at a temperature and pressure such that the porous membrane and thermoplastic film reversibly adhere;
(d′) Forming electrode components by etching the electrically conductive surface using photolithography techniques; and
(e′) Removing the porous membrane and electrode components from the thermoplastic-substrate layer.

6. The method of claim 5, wherein the thermoplastic film comprises a wax and polyolefin blend film and/or wherein the substrate is a thermoplastic substrate.

7. The method of claim 1, wherein the porous membrane is formed from a polyester, polycarbonate, polytetrafluoroethylene (PTFE) and/or silicon.

8. The method of claim 7, wherein the porous membrane is a polyethylene terephthalate membrane.

9. The method of claim 1, wherein the porous membrane has a pore size between 0.1 μm and 20 μm.

10. The method of claim 1, wherein the electrodes are formed of gold or a biocompatible gold alloy.

11. The method of claim 1, wherein the electrodes each have a diameter between 125 μm and 2.5 mm.

12. The method of claim 5, wherein the porous membrane is formed from a polyester, polycarbonate, polytetrafluoroethylene (PTFE) and/or silicon.

13. The method of claim 12, wherein the porous membrane is a polyethylene terephthalate membrane.

14. The method of claim 5, wherein the porous membrane has a pore size between 0.1 μm and 20 μm.

15. The method of claim 5, wherein the electrodes each have a diameter between 125 μm and 2.5 mm.

16. An electrical cell-substrate impedance sensing (ECIS) membrane electrode produced by the method of claim 1.

17. An electrical cell-substrate impedance sensing (ECIS) membrane electrode produced by the method of claim 5.
